(19) United States
(12) Patent Application Publication
(10) Pub. No.: US 2014/0123330 A1
(43) Pub. Date: May 1, 2014

(54) CONTROL OF SEXUAL MATURATION IN ANIMALS

(71) Applicant: RECOMBINETICS, INC., SAINT PAUL, MN (US)

(72) Inventors: Daniel F. Carlson, Inver Grove Heights, MN (US); Scott C. Fahrenkrug, Minneapolis, MN (US); Xavier Lauth, San Diego, CA (US)

(21) Appl. No.: 14/067,634

(22) Filed: Oct. 30, 2013

Related U.S. Application Data
(60) Provisional application No. 61/870,510, filed on Aug. 27, 2013.
(60) Provisional application No. 61/720,187, filed on Oct. 30, 2012.

(51) Int. Cl. C12N 15/85; A01K 67/027

(52) U.S. Cl. CPC C12N 15/8509; A01K 67/0276
USPC 800/15; 435/325; 435/349; 435/352; 514/9.8; 800/13; 800/14; 800/16; 800/17; 800/19; 800/20; 800/21

(57) ABSTRACT

A genetically modified livestock animal comprising a genome that comprises inactivation of a neuroendocrine gene selective for sexual maturation, with the inactivation of the gene preventing the animal from becoming sexually mature. Methods of using, and processes of making, the animals are taught.

### CROSS REFERENCE TO RELATED APPLICATIONS

This patent application claims priority to U.S. Provisional Application No. 61/720,187 filed Oct. 30, 2012 and U.S. Provisional Application No. 61/870,510 filed Aug. 27, 2013, each of which are hereby incorporated by reference herein.

### STATEMENT AS TO FEDERALLY SPONSORED RESEARCH

Aspects of the work described herein were supported by grant 1R43RR033149-01A1 from the National Institutes of Health and Biotechnology Risk Assessment Program competitive grant number 2012-33522-19766 from the USDA-National Institute of Food and Agriculture. The United States Government may have certain rights in these inventions.

### TECHNICAL FIELD

The technical field relates to methods of making and raising animals, including livestock, with the livestock being genetically modified so that they remain sexually immature unless treated to become mature.

### BACKGROUND

Conventional livestock raising practices focus on the role of sexual maturity in livestock in terms of optimizing breeding and parturition. With a herd of cows, for instance, management of a heifer during the sexual maturation period is known to significantly affect her lifetime productivity and should be carefully planned. The optimum process for sexual development has been the object of much research, with conventional wisdom being the idea that heifers that breed and calve early their first year have better lifetime production as well as a reduction in overall production costs up to the initial calving.

### SUMMARY

Processes are provided herein that, in contrast to conventional practices, delay livestock sexual maturity indefinitely, permanently, or until such time as they are treated to become sexually mature. Fish and swine have been treated with these processes, with the results of these treatments being set forth herein, including live, cloned, founder animals. High-efficiency and precise gene-editing was achieved in certain commercially important loci. Efficiencies are high enough so that these changes can be made without linked selection markers.

An embodiment of the invention is a genetically modified livestock animal comprising a genome that comprises inactivation of a neuroendocrine gene selective for sexual maturation, with the inactivation of the gene preventing the animal from becoming sexually mature.

An embodiment of the invention is a genetically modified livestock animal comprising a genome that is heterozygous for an inactivation of a neuroendocrine gene selective for sexual maturation, wherein progeny homozygous for the inactivated gene are thereby prevented from becoming sexually mature.

An embodiment of the invention is a process of making a livestock animal comprising introducing, into an organism chosen from the group consisting of a livestock cell and a livestock embryo, an agent that specifically binds to a chromosomal target site of the cell and causes a double-stranded DNA break to inactivate a neuroendocrine gene selective for sexual maturation, with the agent being chosen from the group consisting of a TALEN, a zinc finger nuclease, and a recombinase fusion protein.

An embodiment of the invention is a process of raising a livestock animal comprising administering an agent to an animal for sexual maturation of the animal, with the agent compensating for a genetic inability of the animal to sexually mature.

The following patent applications are hereby incorporated herein by reference for all purposes; in case of conflict, the instant specification is controlling: US2010/0146655, US2010/0105140, US2011/0059160, US2011/0197290, and US2013/0117870.

### BRIEF DESCRIPTION OF THE DRAWINGS

FIG. 1. is an illustration of a process of making and using animals genetically modified for control of maturation.

FIG. 2. Confirmation of Belgian Blue introgression by sequencing. The schematics of Wagyu wild-type GDF8 and the Belgian Blue template (BB-HDR) are shown. PCR was conducted using primers located outside of the homology arms (c and d) on five PCR positive colonies followed by cloning and sequencing with primer b′. Comparison to the wild-type sequence revealed the expected 11-basepair deletion characteristic the Belgian Blue allele (heterozygous) in 4 of 5 colonies.

FIG. 3. Introgression of naturally occurring alleles from one species to another using mRNA encoded TALENs and ssODNs. The Piedmontese Myostatin allele C313Y was introgressed into Ossabaw.

FIG. 4. Modification of targeted genes. Each chart displays results of targeting a specific locus in fibroblasts (e.g., ssIL2RG; “ss” for Sus scrofa and “bt” for Bos taurus). (Insets) Diagrams of the oligo templates, in which the shaded boxes represent the TALEN-binding site and the spacers are shown in white. HDR was measured at days 3 and 10 by RFLP analysis (Day 3 % HDR and Day 10 % HDR). Each bar displays the average and SEM from three replicates.

FIG. 5. Sequence analysis of TALEN stimulated HDR alleles. PCR amplicons flanking the target site (200-250 bp total) derived from TALEN mRNA and oligo transfected cell populations were sequenced by ILLUMINA sequencing. Total read count ranged from 10,000 to 400,000 per sample. The count of perfect, intended HR reads versus the wild type reads is plotted for insertion (panel a) and SNP alleles (panel b). The target locus, time point and whether or not BMs were included in the oligo are indicated below. Panel c). Reads from btGDF8 and p65 were sorted for incorporation of the target SNP and then classified intended (iSNP) versus those with an additional mutation (iSNP+Mut) and plotted against the total number of reads.

FIG. 6. Cloned pigs with HDR alleles of DAZL and APC. (A) RFLP analysis of cloned piglets derived from DAZL- and APC-modified landrace and Ossabaw fibroblasts, respectively. Expected RFLP products for DAZL founders are 312, 242, and 70 bp (open triangles), and those for APC are 310, 221, and 89 bp (filled triangles). The difference in size of the 312-bp band between WT and DAZL founders reflects the expected deletion alleles. (B) Sequence analysis confirming the presence of the HDR allele in three of eight DAZL founders, and in six of six APC founders. BMs in the donor templates (HDR) are indicated with arrows, and inserted bases are enclosed in blocks. The bold text in the top WT sequence indicates the TALEN-binding sites.

FIG. 7 A schematic of porcine GPR54 and the gene targeting strategy for knockout is depicted in panel a. TALENs designed to bind exon 3 (underlined text) were co-transfected with an oligonucleotide homology template (HDR) designed to introduce a premature stop codon and a HindIII restriction site. Panel b: 2 micrograms of TALENs encoding mRNA plus 0.2 nMol of the HDR template were transfected into pig fibroblasts that were grown as colonies and analyzed for homology dependent repair by HindIII RFLP assay. PCR results are shown; each lane represents one colony. Cleavage products of 231 and 158 bp are indicative of homology dependent repair. Colonies with the parent band of 389 bp are classified as heterozygous (open triangle) and those without are classified as homozygous (filled triangle) for the HDR, knockout allele.

FIG. 8 Panel a: Nucleotide and deduced translated amino acid sequence of mRNA encoding tilapia kisspeptin. The structural organization of the kiss gene is conserved and contains two coding exons, one encoding both the signal peptide and part of the kisspeptin precursor, the other encoding the remainder of the precursor including the kisspeptin-10 sequence. The position of the intron is indicated by a triangle glyph. The location of the forward and reverse primers for PCR amplification of the target region (442 bp) are shown. The binding sites for the two engineered pairs of TALENs, Kiss1.1a and Kiss1.1b are indicated in black and gray boxes. Panel b shows a schematic representation of the targeted kiss genomic region showing the location of the kisspeptin-10 biologically active peptide and each kiss1.1a and 1b TALENs recognition sites. PCR (442 bp) and qPCR primer pairs (138 bp amplicon) used for analysis of indels are shown as well.

FIG. 9 Panel a: Nucleotide and deduced translated amino acid sequence of mRNA encoding tilapia GPR-24 mRNA. The structural organization of the kissr gene is conserved and contains five coding exons. The positions of all four introns are indicated by a triangle glyph. The KissRE2 and KissRE3 TALENs targeted loci are located in the coding exon 2 (white boxes) and 3 (gray boxes) respectively. The location of the sense Left and antisense Right TALENs recognition sites are shown in boxes. Panel b shows a schematic representation of the tilapia GPR-24 genomic region showing the location of the introns (Stroked goalpost), the coding exons 2 and 3 (black arrows) containing the kissRE2 and RE3 loci (white boxes). The location primers used for PCR and qPCR analysis and the size of the corresponding amplicons are shown as well.

FIG. 10 Melt analysis of 100-120 bp qPCR product containing the kiss and kissRE3 loci. Panels a and b show melting curves of amplicons generated from the gDNA extracted from the fin of fish treated kiss1.1a and kissRE3 TALENs pairs. The plain arrows point to melting profiles (panel a) or (panel b) that were significantly different than those obtained from untreated fish (dotted arrows) and correspond to candidate mutant fish kiss #41, RE3 #1, 4, 6 and 11. Panel c: A 442 bp genomic segment containing the targeted Kiss loci was PCR amplified from—TALEN treated fish #41. The PCR product was cloned into TOPO 2.1 TA vector, and transformant colonies were hand-picked for direct QPCR analysis. The plain arrows point to selected melt profiles obtained from colonies containing different deletions at the kiss loci. Panel d: To better visualized the varied mutations cloned, we graphed our QPCR colony screen on a scatter plot of Cts versus melt temperature, where each clone is represented by a data plot (x, y) with x representing its Ct and y representing its melt temperature. The graph represent colonies containing the 702 bp PCR fragment amplified from Fish RE3 #4. Melt temperature below that of a wild type sequence all contained the kissRE3 amplicon with varied deletions at the target site. Cts: Cycle thresholds.

FIG. 11: Description of somatic mutations induced by engineered TALENs at the kiss gene (site kiss1.1a) (panel a) and kissR gene site (KissRE3) (panel b). The wild-type sequences are shown at the top of each panel with the sense left and antisense right TALEN recognition element sites shown in bold highlighted in dark gray and the sense spacer highlighted as underlined text. Deletions are shown as dashes and insertions as lower case letters highlighted in light gray. The net change in length caused by each indel mutation is to the right of each sequence (+, insertion; −, deletion). A few alterations have both a deletion and an insertion of sequence. The number of times each mutant allele was isolated is shown in brackets.

FIG. 12: Panel a: Selected sequencing chromatography of PCR products from two sibling progeny in line KissRE3 #11. These graphs indicate the presence of mutation reading simultaneously a kissRE3 mutant and a WT allele. Boxes indicate matching nucleotides on the mutant and WT alleles and arrow points to the location where sequences become divergent and thus where these deletion begin. To characterize the mutation we analyzed the pattern of unique nucleotide reads in the sequence (where the chromatograph show above background non duplicate nucleotide reads). By shifting the WT sequence and increased size deletion sequences, we found that a 7 bp and 5 bp deletions reproduce the pattern of single nucleotide reads on these chromatograph. Panel b: Description of all inherited indel mutations induced by engineered TALENs at the kiss gene (kiss1.1a site, top) and kissr gene (KissRE3 site, bottom). The wild-type sequences are shown at the top with the sense left and antisense right TALEN recognition elements shown in bold letter highlighted in dark gray and the sense spacer highlighted as underlined text. Deletions are shown as dashes. The net change in length caused by each indel mutation is to the right of each sequence (−, deletion). The number of times each mutant allele was isolated is shown in brackets. Panel c: Description of the most severe lesions found at the kiss and kissRE3 sites. The 18 nt deletion at the kiss1.1a loci result in the loss of 6AA (underlined) 3 of which are from the core sequence of the kisspeptin-10 active peptide (highlighted in gray). The Int deletion at the kissRE3 loci (underlined text) result in significant alteration of the gene product with two AA substitution immediately followed by a stop codon. The resulting protein is C-terminally truncated by 215 AA.

### DETAILED DESCRIPTION

It is desirable to produce livestock in a way that conserves environmental and energy resources. Sexually immature animals generally consume less food per pound of weight than mature or maturing animals. Livestock, in general, do not reach a desirable size before maturation. Set forth herein, however, are animals that can be grown to a desirable size before maturation.

In fact, methods are described herein whereby an animal does not sexually mature at all. It can be grown past the normal age of maturity without passing through pubescence. Sexually immature animals are sterile. The efficient production of sterile animals is therefore a significant challenge since sexual reproduction is cost effective, and even assisted reproductive techniques (ARTs) require a mature animal to provide ova and sperm. In some embodiments, the livestock animal does not pass into puberty and remains permanently sexually immature unless specifically treated to allow it to pass into sexual maturity. Such animals, after treatment to induce maturity, can then be bred.

An advantage of making livestock incapable of maturing is that they are unable to reproduce. In the case of sexually-bred or genetically modified fish, for instance, concerns about their accidental release into the wild are eliminated. Other animals that are similarly modified will also be unable to reproduce, so that animals with valuable genetic traits can be sold without concerns of uncontrolled breeding of the animals by the buyers. Further, in many farm animals (e.g., cows, poultry, and fish) sterilization will increase productivity as well as meat quality, improvements in lipid content, pigmentation and texture. The term cow is a colloquial term for cattle; cattle are large ungulates, are the most widespread species of the genus Bos. A cow or cattle refers to a member of Bos primigenius. And, in the case of fish, sterile fish should demonstrate greater performance in culture by conserving energy for growth rather than gonad development and sexual differentiation. Currently, sterilization through ploidy manipulation (specifically triploidy, which adds of one extra set of chromosomes) is the only commercially scalable technique available to aquaculture producers. However, inconsistent results have raised concerns with respect to the efficacy of the technique. In addition, triploid induction, in general, often negatively impacts survival and/or performance of treated populations. And the application of the technology is labor intensive, logistically complicated and costly.

An embodiment of the invention is a genetically modified livestock animal comprising a genome that comprises an inactivation of a neuroendocrine gene selective for sexual maturation, with the inactivation of the gene preventing the animal from becoming sexually mature. The gene is selectively directed to sexual maturation processes and, if knocked-out of an animal, the animal will be comparable to wild-type animals in terms of its development as measured by size and weight until such time as the wild type animals undergo sexual maturation. The term gene means a locatable region of genomic sequence, corresponding to a unit of inheritance, which is associated with regulatory regions, transcribed regions, and or other functional sequence regions. The term gene, as used herein, includes the functional sequence regions as well as those portions that encode a protein or other factor. The term knocked-out, as used herein, refers to the direct or indirect disruption of a gene that either inactivates function in the resulting protein or eliminates production of the protein product.

Since the genetic modifications are directed to a specific gene or gene product to prevent sexual maturation, the factor that is needed for maturation is known and can generally be supplied.

### Neuroendocrine Genes Selective for Sexual Maturation

Sexual development of animals may be prevented by blocking neuroendocrine genes selective for sexual maturation. Sexual development, accelerated growth, and adrenal maturation is initiated when gonadotropin-releasing hormone (GnRH1) begins to be secreted by the hypothalamus. The gene GnRH1 encodes the GnRH11 precursor. In mammals, the linear decapeptide end-product is generally synthesized from a 92-amino acid preprohormone. Gonadotropin-releasing hormone (GnRH1), also known as Luteinizing-hormone-releasing hormone (LHRH) and luliberin, is responsible for the release of follicle-stimulating hormone (FSH) and luteinizing hormone (LH). GnRH1 belongs to gonadotropin-releasing hormone family. Embodiments of the invention include inactivating GnRH1 in a livestock animal. Gonadotropin-releasing hormone or analogues may be administered to the animal to bring it to sexual maturity. Sequences for GnRH1 across multiple species are well known, e.g., Gene IDs 768325 for Bos Taurus, 770134 for Gallus gallus, or 397516 for sus scrofa. GPR54, also known as the Kisspeptin receptor (also referred to as GpR54, KissR, Kiss1R, kissR and the like), binds to the hormone Kisspeptin (formerly known as metastin). Kisspeptin is a product derived from the KiSS1 gene (also referred to as Kiss, Kiss1, KiSS, kiss1 and the like). Kisspeptin-GPR54 signaling has a role in initiating secretion of GnRH1. Kisspeptin is an RFamide neuropeptide with multiple functions, involving varied whole body physiological systems and acting at all levels of the reproductive axis—brain, pituitary, gonad (BPG), and accessory organs. Kisspeptin can directly stimulate GnRH release (Messager et al., 2005), relaying steroid hormone negative and positive feedback signals to GnRH neurons, serving as a gatekeeper to the onset of puberty, and relaying photoperiodic information.

Embodiments of the invention include inactivating the gene GPR54 and/or KiSS1 in a livestock animal. Kisspeptin may be administered to make-up for a loss of KiSS1 and thereby achieve sexual maturity. Or, KiSS1 and/or GPR54 is suppressed, and gonadotropin-releasing hormone may be administered to the animal to bring it to sexual maturity. Another embodiment is inactivation of the Kisspeptin-GPR54 interaction by inserting a dominant negative GPR54 into the genome of a livestock animal. Expression of the dominant negative GPR54 prevents initiation of sexual maturation. Expression of the dominant negative GPR54 interferes with signal transduction downstream of the receptor, preventing signal propagation and release of GnRH1. Sequences for GPR54 are well known across multiple species, e.g., 84634 for Homo sapiens, 561898 for Danio rerio, or 733704 for Sus scrofa. Sequences for Kiss1 are well known across multiple species, e.g., 615613 for Bos taurus, 733704 for Sus scrofa, or 100294562 for Ovis cries.

The Gpr54/Kiss pathway is highly conserved among most vertebrate species and is known to be the gatekeeper to puberty in humans and mice. (Seminara et al., 2003). Infertility due to inactivation of the Gpr54 and/or Kiss gene in humans and mice has been reverted by ectopic GnRH administration. Studies in mice and humans demonstrate that inactivation Gpr54 effectively leads to infertility of both sexes due to hypogonadotropic hypogonadism (d'Anglemont de Tassigny et al., 2007; de Roux et al., 2003). The Kiss-Gpr54 system is highly conserved in vertebrates (Tena-Sempere et al., 2012) particularly in mammals where only one Kiss and Gpr54 gene is present. Whereas multiple distinct Kiss genes have been identified in fish, the receptor Gpr54 is encoded by one gene in all but one species examined. Humans and mice with Gpr54 mutations displayed normal levels of hypothalamic GnRH suggesting Kiss/Gpr54 signaling was responsible for the release of GnRH into the blood stream (Seminara et al., 2003). This presented an opportunity to bypass Kiss/Gpr54 signaling by injection of GnRH or gonadotropins directly into Gpr54-deficient subjects. Indeed, both Gpr54-deficient humans and were responsive to GnRH injection (Seminara et al., 2003) indicating that downstream signaling components of puberty remain intact.

There is currently no direct evidence of a piscine kisspeptin role in reproductive biology publications. However, administration of kiss peptide has been shown to stimulate gonadotropin gene expression in the pituitary of sexually mature female zebrafish (Kitahashi et al. 2008) and orange grouper, or secretion of LH and FSH in European sea bass (Felip et al., 2008) and goldfish. Thus, in theory, the fertility of sexually immature and sterile fish with knockouts of GPR54 and/or KiSS1 can be rescued by exogenous delivery of kisspeptin analogues (e.g., Kisspeptin 10) or gonadotropin analogues (LH or FSH). With this concept, homozygous kiss or kiss receptor knockout-broodstock can be bred in captivity if administered the corrective hormone, ensuring reversible control over fertility. The progeny from this KO-broodstock inherits the alteration. This would provide economic and environmental benefit.

Neuroendocrine genes selective for sexual maturation can be inactivated by a number of processes. Inactivation of the gene prevents expression of a functional factor encoded by the gene, such as a protein or an RNA. One kind of inactivation comprises an insertion, deletion, or substitution of one or more bases in a sequence encoding the sexual maturation factor and/or a promoter and/or an operator that is necessary for expression of the factor in the animal. The inactivation may be a knock-out of a gene. The gene may be inactivated by removal of at least a portion of the gene from a genome of the animal, alteration of the gene to prevent expression of a functional factor encoded by the gene, an interfering RNA (expressed by a gene in a genome of the animal or in a plurality of cells of the animal), or expression of a dominant negative factor by an exogenous gene.

Another system for revertible-infertility is Tac3/TacR3 (Young, J., Bouligand, J., Francou, B., Raffin-Sanson, M. L., Gaillez, S., Jeanpierre, M., Grynberg, M., Kamenicky, P., Chanson, P., Brailly-Tabard, S., et al. (2010). TAC3 and TACR3 defects cause hypothalamic congenital hypogonadotropic hypogonadism in humans. J Clin Endocrinol Metab 95, 2287-2295. As with Kiss/Gpr54, humans deficient for these genes display hypogonadotropic hypogonadism which was revertible by pulsatile GnRH treatment (Young et al., 2010). Tac and/or Tac3 may be inactivated using methods described or referenced herein.

Embodiments of the invention include methods of inactivating one or more genes selected from the group consisting of GnRH1, GPR54, KiSS1, Tac and Tac3 in animals selected from the group consisting of cattle, sheep, pigs, chickens, turkeys, goats, sheep, fish, buffalo, emu, rabbits, ungulates, avians, rodents, and livestock. The genes may be inactivated in cells and/or embryos and in animals resulting therefrom. Various methods are described herein, e.g., knocking out a gene in a cell or embryo using TALENs or Zinc Finger Nucleases, and cloning and/or implanting the cell/embryo in a surrogate to make a founder animal.

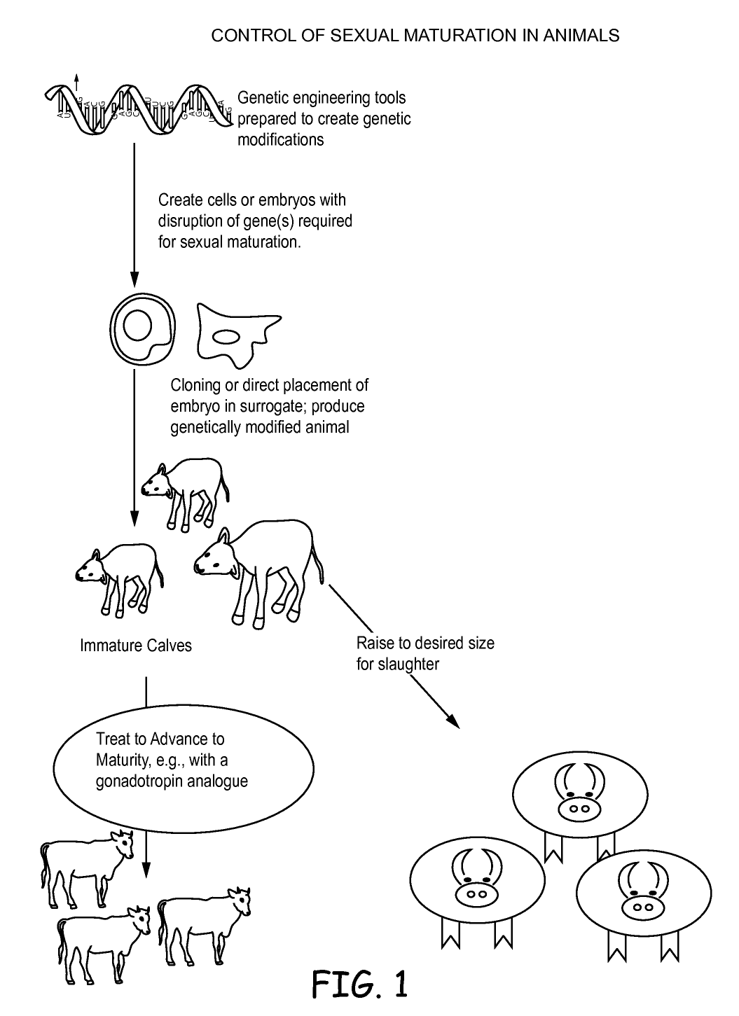

FIG. 1 illustrates an embodiment of the invention, with bovine cells being modified in vitro and used to clone calves. The calves may be raised to a suitable weight for slaughter or treated with factors that allow them to pass into maturity, e.g., gonadotropin analogues or a factor that directly supplied a knocked-out genetic factor.

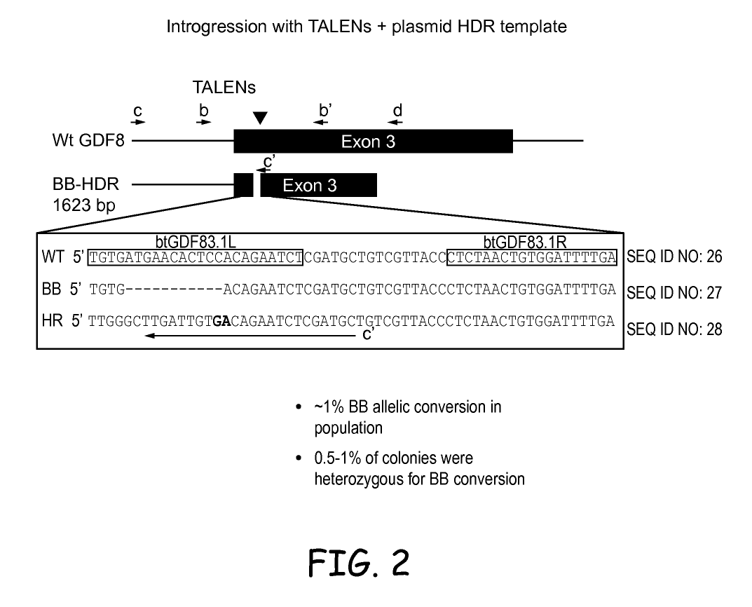

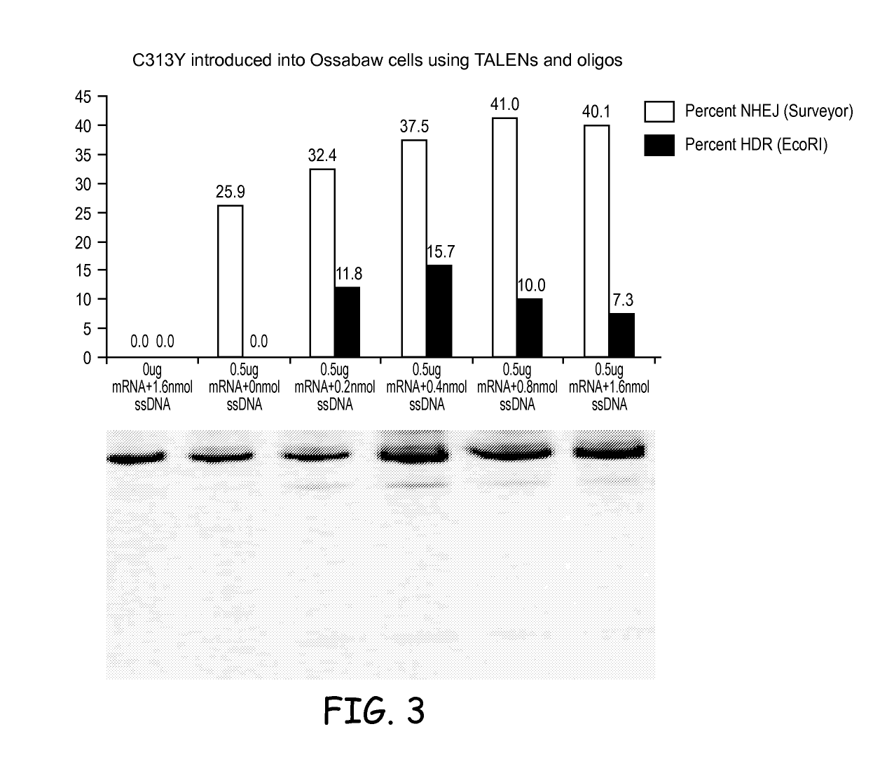

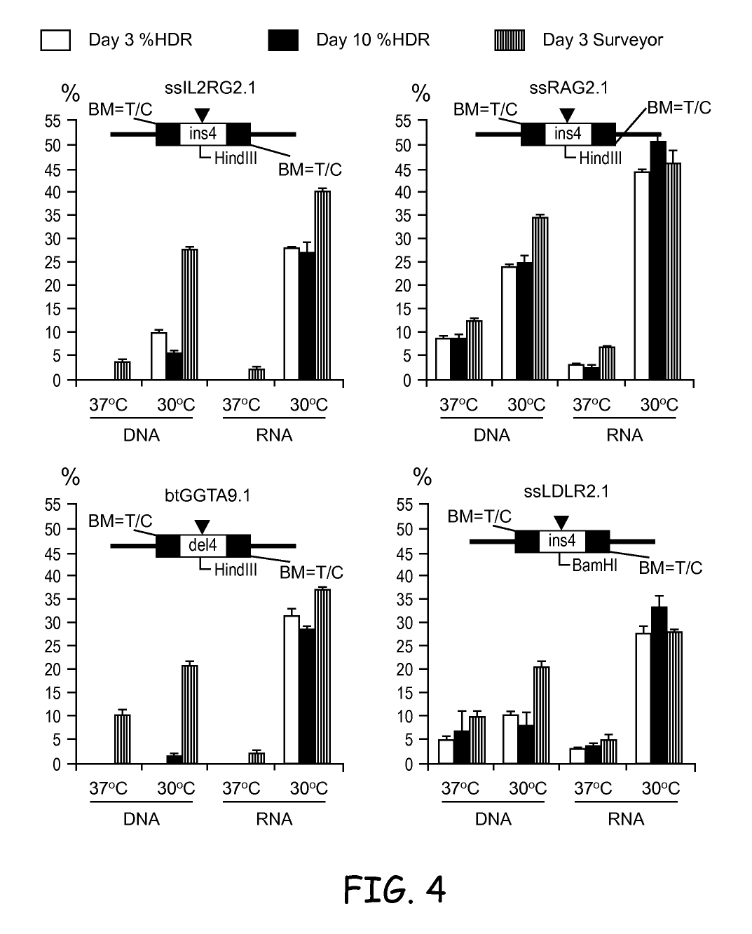

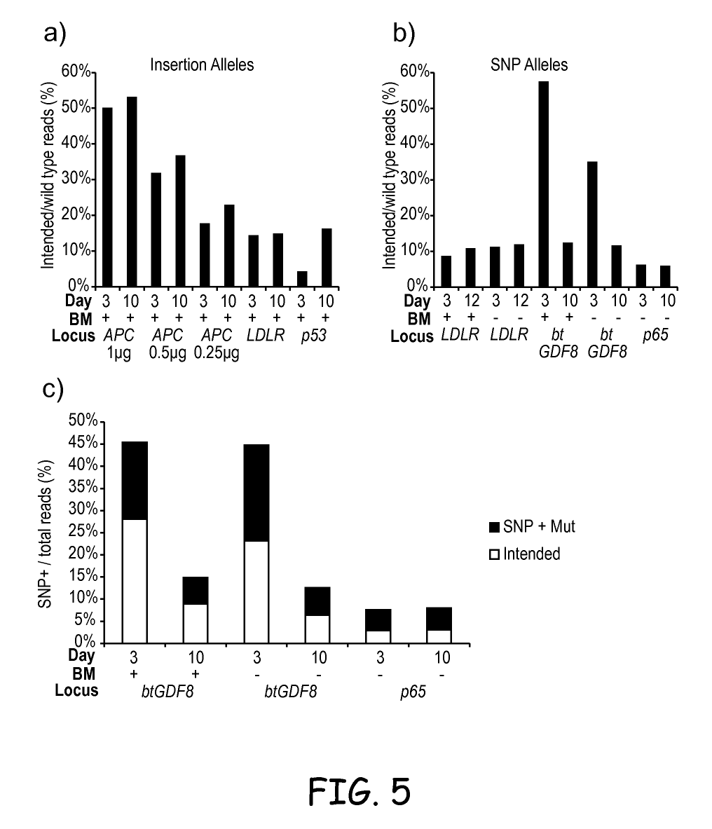

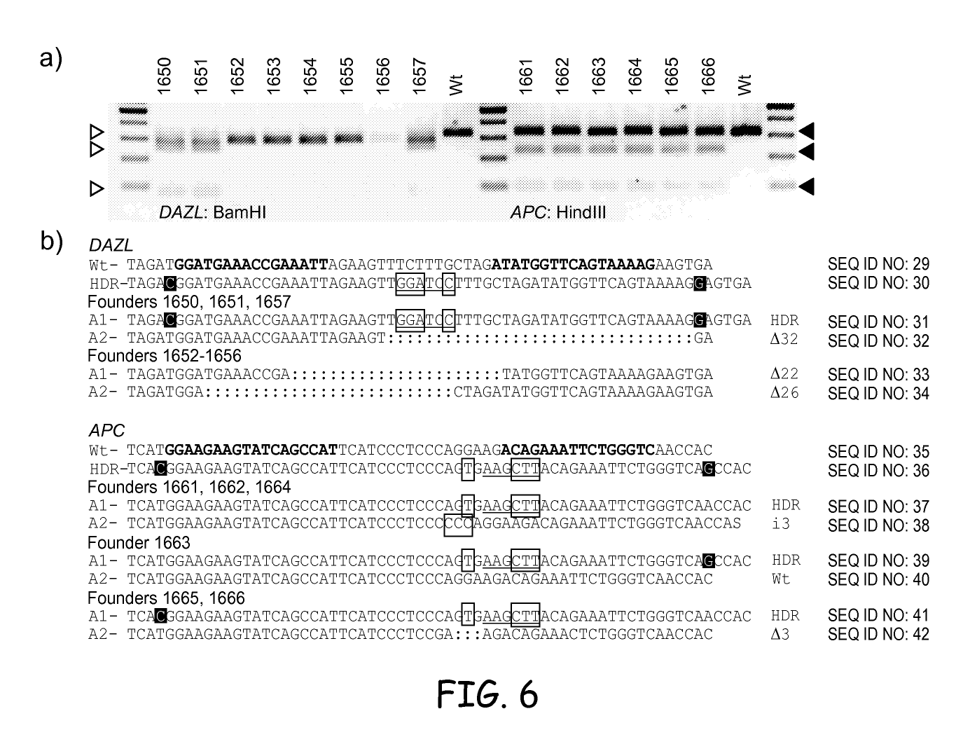

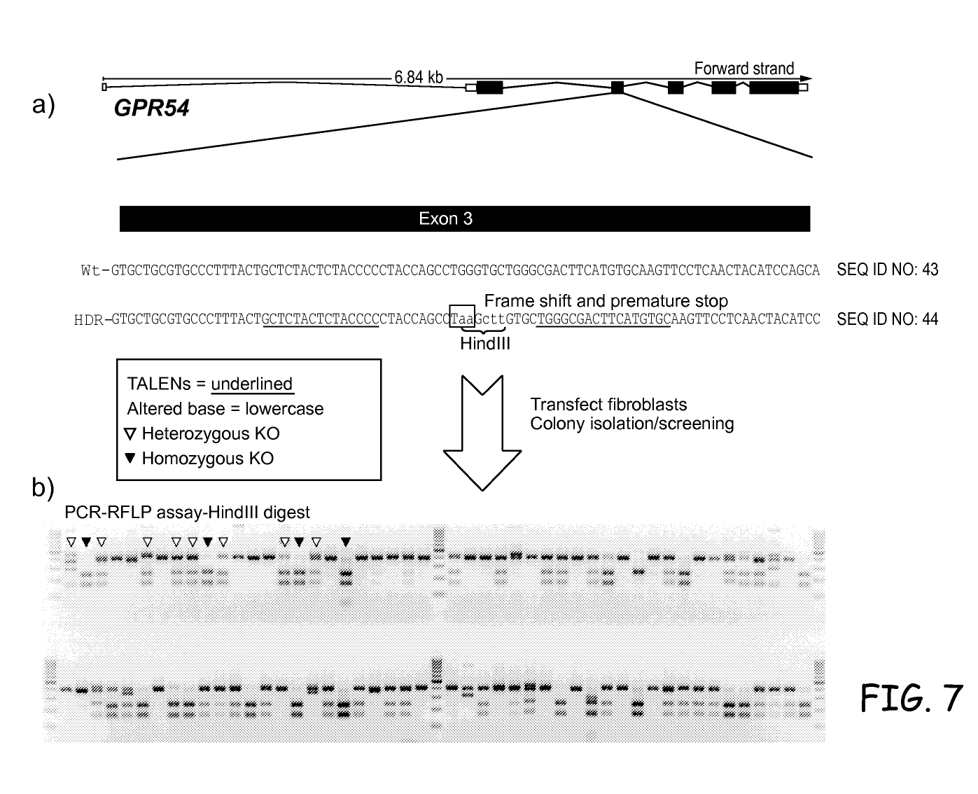

Example 1 describes techniques for making changes to cells with a TALENs system. Example 2 describes the dilution cloning technique used for the results of Table 1. Example 3 describes the techniques of mutation detection and RFLP analysis. Example 4 (FIG. 2.) describes introgression of an 11-basepair deletion into exon-3 of bovine GDF8 (Belgium Blue mutation). FIG. 3 depicts results for a similar process that introgressed an allele from one species into another species. Example 5 describes testing for the same as well as introgression of alleles into cow cells using oligo HDR. In Example 5, TALEN-induced homologous recombination eliminated the need for linked selection markers. When transfected alone, the btGDF8.1 TALEN pair cleaved up to 16% of chromosomes at the target locus. Co-transfection with a supercoiled homologous DNA repair template harboring the 11 bp deletion resulted in a gene conversion frequency (HDR) of 0.5 to 5% at day 3 without selection for the desired event. Gene conversion was identified in 1.4% of isolated colonies that were screened by PCR, which was a rapid method to identify successful alterations. Example 6 (FIG. 4) describes the modification of four intended loci in pig and cattle fibroblasts. Example 7 (FIG. 5) shows analysis of modifications made to genes APC, LDLR, p53, p65, and btGDF8. Example 8 (Table 1) shows a recovery rate for intended indels of 10-64% (average, 45%), with up to 32% of the colonies homozygous for the modification. Example 9 (FIG. 6) describes cloned pigs that were made with modified deleted in azoospermia-like (DAZL) and modified adenomatous polyposis coli (APC) genes. Example 10 (FIG. 7) describes GPR54 knockouts, made according to the indicated gene targeting strategy; Example 11 details methods for making modified animals with the GPR54 knockout. Example 12 describes modifications made with custom-made CRISPR/Cas9 endonucleases.

These results demonstrated techniques that effective make modifications at an intended gene, without the aid of a linked selection marker. Cells with the modifications can be used for cloning animals. The intended genetic modifications can be controlled with specificity, for instance, for introgressing an allele or to modify a gene. Modifications may be, for instance, a deletion or an insertion to disrupt a gene or knock it out, or to replace part of the gene to make a nonfunctional gene product or an alternative product.

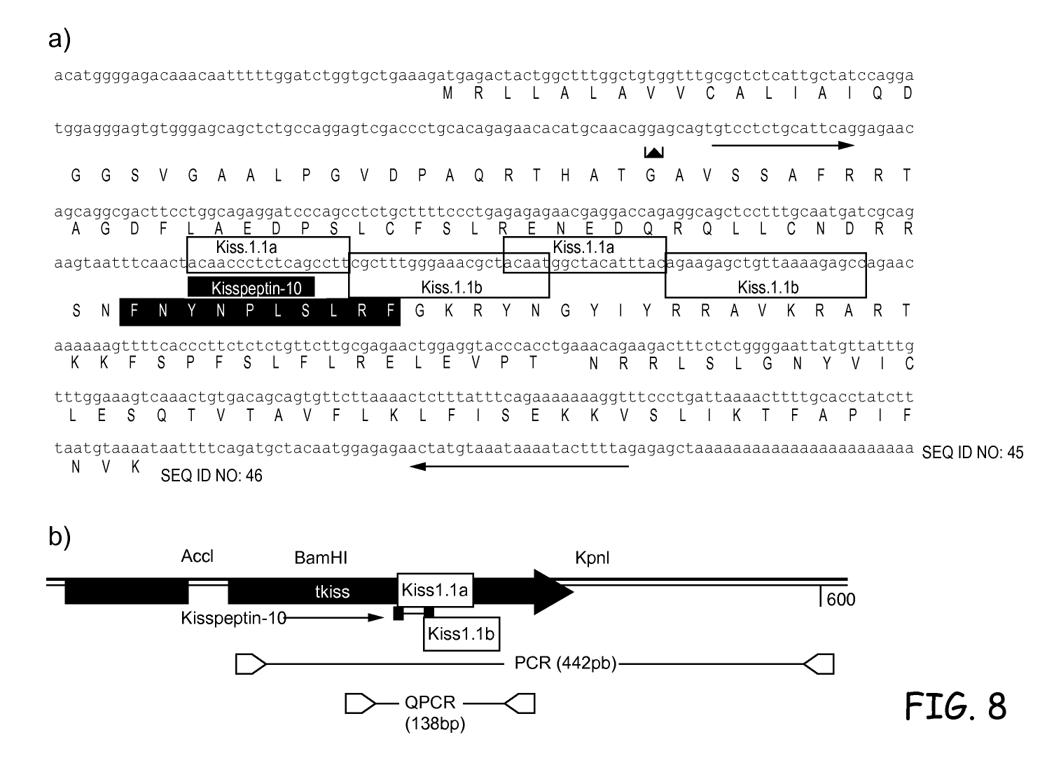

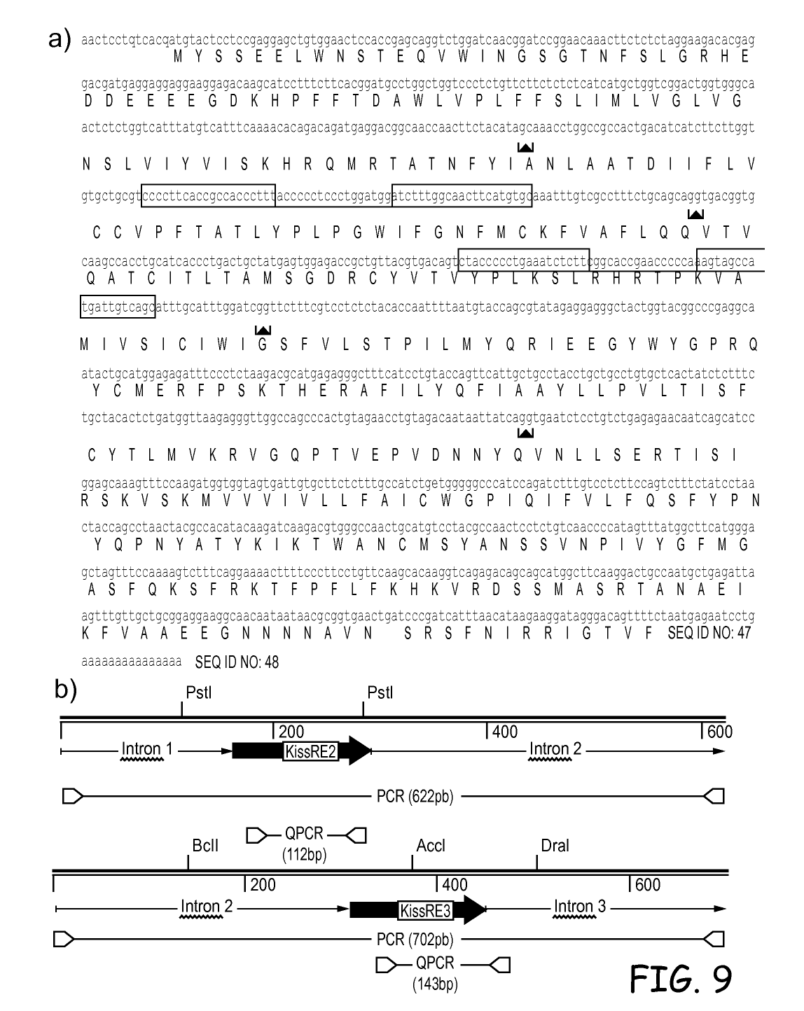

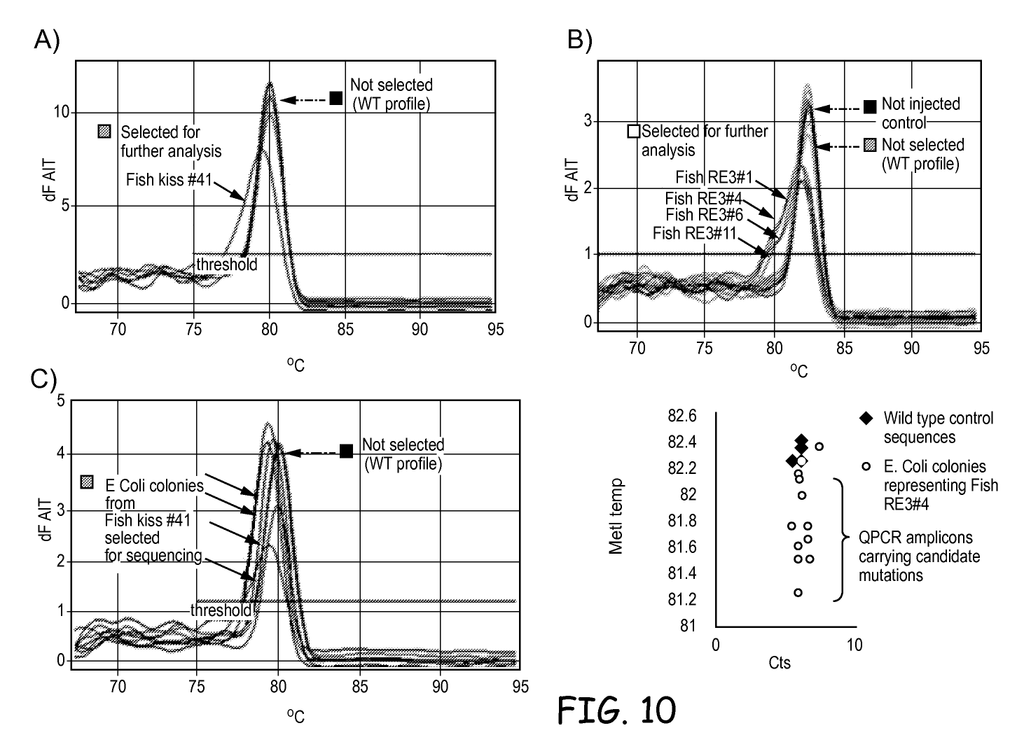

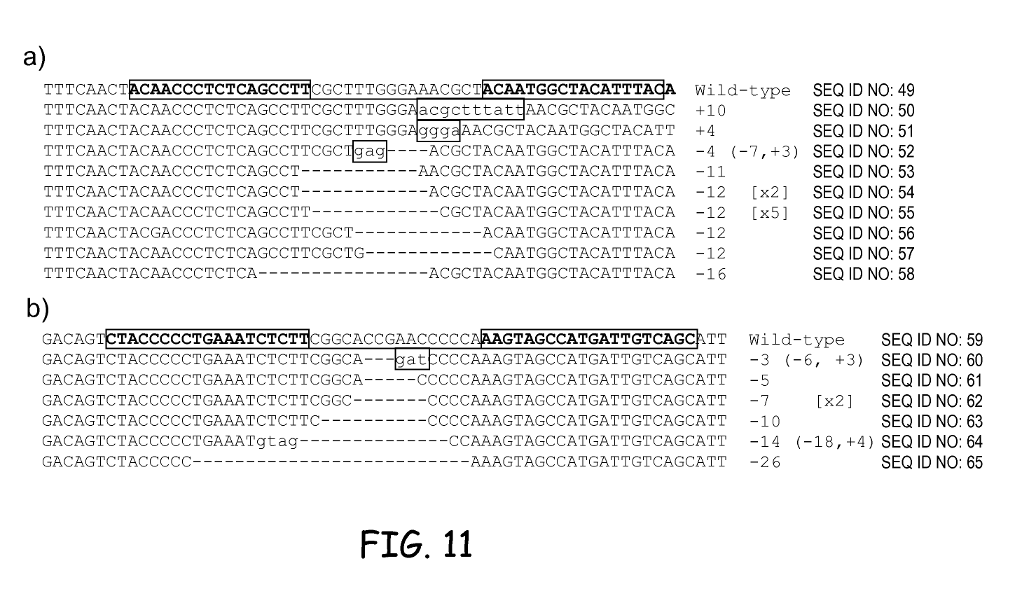

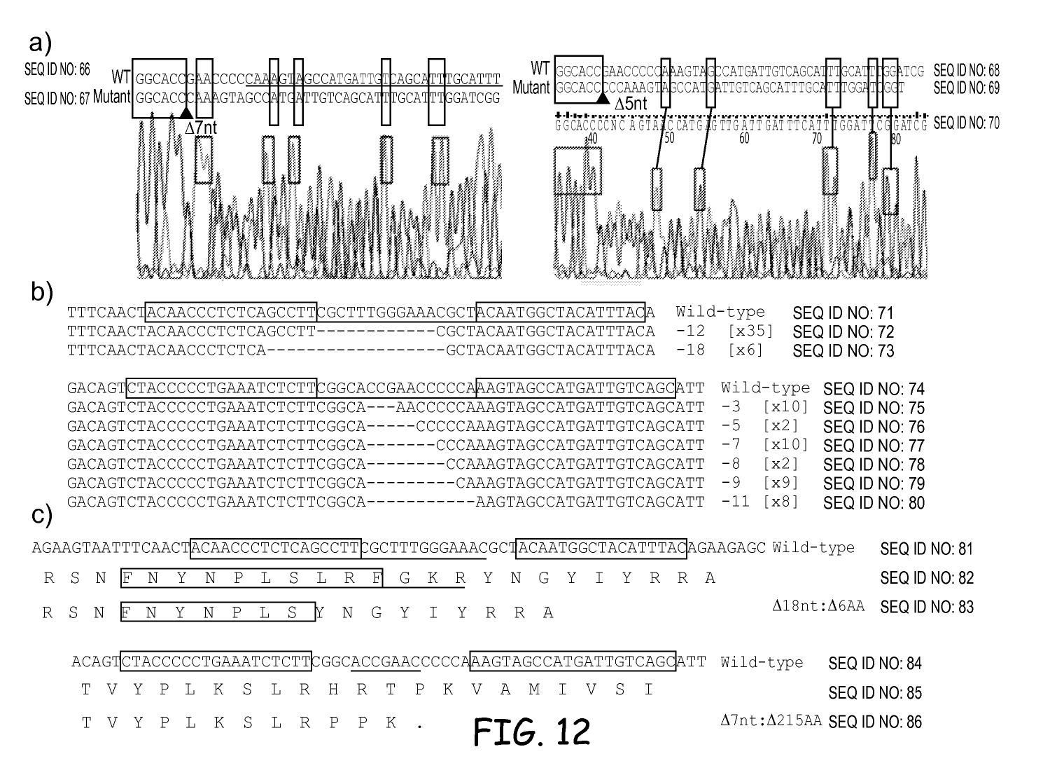

Fish (tilapia) with a knockout of KiSS1 and GpR54 (also referred to as GPR54, Kiss-receptor, KissR, Kiss1R) have been made. FIGS. 8 and 9 depict the targeted regions for KISS and GpR54. The structural organization of the Kiss gene is conserved and contains two coding exons, one encoding both the signal peptide and part of the kisspeptin precursor, the other encoding the remainder of the precursor including the kisspeptin-10 sequence. Example 14 details the steps that were used to make founder fish with Kiss or KissR knockouts. Techniques based on TALENs were used to knock out the genes and melt analysis was used to detect indels (FIG. 10). Various modifications at the targeted genes were confirmed (FIG. 11), including nine different nucleotide deletions, two insertions, and three combinations of nucleotide insertions and deletions. Sequencing indicated that a knockout would result from at least some of these modifications. Germ line mutations were confirmed (see FIG. 12). F1 heterozygous mutants with a Kiss or KissR knockout were created and bred. F2 generations, which are expected to show the inactivation phenotype, are presently being grown.

Disclosed herein are processes to make transgenic animals that have changes only at an intended site. Additionally, the processes can make specifically intended changes at the intended site. It is not necessary to remove other changes resulting from problems like the use of linked-reporter genes, or linked positive and negative selection genes, or random transgene integration are bypassed. Moreover, the processes can be used in the founder generation to make genetically modified animals that have only the intended change at the intended site. Other processes are also disclosed that involve unlinked marker genes and the like.

### Targeted Nuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making indels with precision at an intended residue, and placement of the allele that is being introgressed at the DNA-binding site.

### TALENs

TALENs are genetic engineering tools. Inactivation of a gene is one of many uses of TALENs. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA.

One of the barriers to making TALEN-modified livestock is that the efficiency of making a modification to an animal cell is only a few percent with conventional best practices. Achievement of a deletion or an insertion at an intended site does not necessarily mean success because it may not actually create the intended effect, such as expressing an exogenous protein or stopping expression of an endogenous protein. Even a low efficiency can be useful for the creation of genetically modified lower animals such as fruit flies or mice because they have short and prolific reproductive cycles that provide for the creating, testing, and screening of hundreds of animals to determine if there are a few that have been successfully modified. These levels of efficiency that are conventionally achieved, however, are not suited to livestock artiodactyls that have much longer gestational times and comparatively few progeny per pregnancy. U.S. Ser. No. 13/404,662 filed Feb. 24, 2012 “Genetically modified animals and methods for making the same”, which is hereby incorporated herein by reference for all purposes (in case of conflict, the specification is controlling) provides certain methods that address these conventional limitations.

Another barrier to using TALENs to modify livestock is that TALEN-mediated modification of DNA in primary cells is difficult because the cells are unstable. U.S. Pub. No. 2011/0197290 filed Feb. 11, 2011 provides useful methods for modifying these cells, and is hereby incorporated herein by reference for all purposes; in case of conflict, the specification is controlling. The term primary cell means a cell isolated from a living animal, wherein the cell has undergone between 0 and 10 replications since its isolation from the tissue. TALENs may be used to make genetically modified artiodactyl primary cells. These modifications are suited to making founders of genetically modified animal lines by cloning. Also described herein are direct-embryonic injections that that may be used to modify zygotes or embryos, with the modified zygotes or embryos being suited to implant into surrogate females for gestation and delivery of founder animal lines.

Miller et al. (Miller et al. (2011) Nature Biotechnol 29:143) reported making TALENs for site-specific nuclease architecture by linking TAL truncation variants to the catalytic domain of FokI nuclease. The resulting TALENs were shown to induce gene modification in immortalized human cells by means of the two major eukaryotic DNA repair pathways, non-homologous end joining (NHEJ) and homology directed repair. The TALENs can be engineered for specific binding. Improvements of the Miller et al. TALENS are described in U.S. Ser. No. 13/594,694 filed Aug. 24, 2012. Specific binding, as that term is commonly used in the biological arts, refers to a molecule that binds to a target with a relatively high affinity compared to non-targets, and generally involves a plurality of non-covalent interactions, such as electrostatic interactions, van der Waals interactions, hydrogen bonding, and the like. Specific binding interactions characterize antibody-antigen binding, enzyme-substrate binding, and specifically binding protein-receptor interactions.

The cipher for TALs has been reported (PCT Application WO 2011/072246) wherein each DNA binding repeat is responsible for recognizing one base pair in the target DNA sequence. The residues may be assembled to target a DNA sequence, with: (a) HD for recognition of C/G; (b) NI for recognition of A/T; (c) NG for recognition of T/A; (d) NS for recognition of C/G or A/T or T/A or G/C; (e) NN for 30 recognition of G/C or A/T; (f) IG for recognition of T/A; (g) N for recognition of C/G; (h) HG for recognition of C/G or T/A; (i) H for recognition of T/A; and (j) NK for recognition of G/C. In brief, a target site for binding of a TALEN is determined and a fusion molecule comprising a nuclease and a series of RVDs that recognize the target site is created. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different.

In some embodiments, a monomeric TALEN can be used. TALEN typically function as dimers across a bipartite recognition site with a spacer, such that two TAL effector domains are each fused to a catalytic domain of the FokI restriction enzyme, the DNA-recognition sites for each resulting TALEN are separated by a spacer sequence, and binding of each TALEN monomer to the recognition site allows FokI to dimerize and create a double-strand break within the spacer. Monomeric TALENs also can be constructed, however, such that single TAL effectors are fused to a nuclease that does not require dimerization to function. One such nuclease, for example, is a single-chain variant of FokI in which the two monomers are expressed as a single polypeptide. Other naturally occurring or engineered monomeric nucleases also can serve this role. The DNA recognition domain used for a monomeric TALEN can be derived from a naturally occurring TAL effector. Alternatively, the DNA recognition domain can be engineered to recognize a specific DNA target. Engineered single-chain TALENs may be easier to construct and deploy, as they require only one engineered DNA recognition domain. A dimeric DNA sequence-specific nuclease can be generated using two different DNA binding domains (e.g., one TAL effector binding domain and one binding domain from another type of molecule). TALENs may function as dimers across a bipartite recognition site with a spacer. This nuclease architecture also can be used for target-specific nucleases generated from, for example, one TALEN monomer and one zinc finger nuclease monomer. In such cases, the DNA recognition sites for the TALEN and zinc finger nuclease monomers can be separated by a spacer of appropriate length. Binding of the two monomers can allow FokI to dimerize and create a double-strand break within the spacer sequence. DNA binding domains other than zinc fingers, such as homeodomains, myb repeats or leucine zippers, also can be fused to FokI and serve as a partner with a TALEN monomer to create a functional nuclease.

In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The spacer of the target sequence can be selected or varied to modulate TALEN specificity and activity. The flexibility in spacer length indicates that spacer length can be chosen to target particular sequences with high specificity. Further, the variation in activity has been observed for different spacer lengths indicating that spacer length can be chosen to achieve a desired level of TALEN activity.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, Hind1II, NotI, BbvCl, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a homing endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csm I, PI-See L PI-Tti L PI-Mtu I, I-Ceu I, I-See IL 1-See III, HO, PI-Civ I, PI-Ctr L PI-Aae I, PI-Bsu I, PI-Dha I, PI-Dra L PI-Mav L PI-Meh I, PI-Mfu L PI-Mga L PI-Mgo I, L PI-Mka L PI-Mle I, PI-Mma I, PI-30 Msh L PI-Msm I, PI-Mth I, PI-Mtu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-MsoI.

A genetic modification made by TALENs or other tools may be, for example, chosen from the list consisting of an insertion, a deletion, insertion of an exogenous nucleic acid fragment, and a substitution. The term “insertion” is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting of livestock, an artiodactyl, cattle, a swine, a sheep, a goat, a chicken, a rabbit, and a fish. The term livestock means domesticated animals that are raised as commodities for food or biological material. The term artiodactyl means a hoofed mammal of the order Artiodactyla, which includes cattle, deer, camels, hippopotamuses, sheep, and goats that have an even number of toes, usually two or sometimes four, on each foot.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

Genetic modification of cells may also include insertion of a reporter. The reporter may be, e.g., a florescent marker, e.g., green fluorescent protein and yellow fluorescent protein. The reporter may be a selection marker, e.g., puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), or xanthin-guanine phosphoribosyltransferase (XGPRT). Vectors for the reporter, selection marker, and/or one or more TALEN may be a plasmid, transposon, transposase, viral, or other vectors, e.g., as detailed herein.

TALENs may be directed to a plurality of DNA sites. The sites may be separated by several thousand or many thousands of base pairs. The DNA can be rejoined by cellular machinery to thereby cause the deletion of the entire region between the sites. Embodiments include, for example, sites separated by a distance between 1-5 megabases or between 50% and 80% of a chromosome, or between about 100 and about 1,000,000 basepairs; artisans will immediately appreciate that all the ranges and values within the explicitly stated ranges are contemplated, e.g., from about 1,000 to about 10,000 basepairs or from about 500 to about 500,000 basepairs. Alternatively, exogenous DNA may be added to the cell or embryo for insertion of the exogenous DNA, or template-driven repair of the DNA between the sites. Modification at a plurality of sites may be used to make genetically modified cells, embryos, artiodactyls, and livestock. One or more genes may be chosen for complete or at least partial deletion, including a sexual maturation gene or a cis-acting factor thereof.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific Fold cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically modified animals are disclosed in, e.g., U.S. Pat. No. 8,106,255 US20120192298, US20110023159, and US20110281306.

### Templated and Non-Templated Repairs

TALENs, zinc finger nucleases, Cas9/CRISPR and recombinase fusion proteins may be used with or without a template. A template is an exogenous DNA added to the cell for cellular repair machinery to use as a guide (template) to repair double stranded breaks (DSB) in DNA. This process is generally referred to as HDR homology directed repair (HDR). Processes without a template involve making DSBs and providing for cellular machinery to make repairs that are less than perfect, so that an insertion or deletion (an indel) is made. The cellular pathway referred to as Non-homologous end joining (NHEJ) typically mediates non-templated repairs of DSBs. The term NHEJ is commonly used to refer to all such non-templated repairs regardless of whether the NHEJ was involved, or an alternative cellular pathway.

### Vectors and Nucleic Acids

A variety of nucleic acids may be introduced into the artiodactyl or other cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7(3):187; and Hyrup et al. (1996) Bioorgan. Med. Chem. 4:5. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, inducible promoters, and promoters responsive or unresponsive to a particular stimulus. Suitable tissue specific promoters can result in preferential expression of a nucleic acid transcript in beta cells and include, for example, the human insulin promoter. Other tissue specific promoters can result in preferential expression in, for example, hepatocytes or heart tissue and can include the albumin or alpha-myosin heavy chain promoters, respectively. In other embodiments, a promoter that facilitates the expression of a nucleic acid molecule without significant tissue or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al. (2001) Hum. Gene Ther. 12:563; and Kiwaki et al. (1996) Hum. Gene Ther. 7:821.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. (1992) 89:6861, for a review of Cre/lox technology, and Brand and Dymecki, Dev. Cell (2004) 6:7. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be methylated using an SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinP1I endonuclease for 1 hour at 37° C. and assaying by agarose gel electrophoresis.

Nucleic acid constructs can be introduced into embryonic, fetal, or adult artiodactyl cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); Tol2 (Kawakami (2007) Genome Biology 8(Suppl.1):S7; Minos (Pavlopoulos et al. (2007) Genome Biology 8(Suppl.1):S2); Hsmar1 (Miskey et al. (2007)) Mol Cell Biol. 27:4589); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Insulator elements also can be included in a nucleic acid construct to maintain expression of the exogenous nucleic acid and to inhibit the unwanted transcription of host genes. See, for example, U.S. Publication No. 2004/0203158. Typically, an insulator element flanks each side of the transcriptional unit and is internal to the inverted repeat of the transposon. Non-limiting examples of insulator elements include the matrix attachment region-(MAR) type insulator elements and border-type insulator elements. See, for example, U.S. Pat. Nos. 6,395,549, 5,731,178, 6,100,448, and 5,610,053, and U.S. Publication No. 2004/0203158.

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### Genetically Modified Animals

Animals may be modified using TALENs, zinc finger nucleases, or other genetic engineering tools, including various vectors that are known. A genetic modification made by such tools may comprise inactivation of a gene. The term inactivation of a gene refers to preventing the formation of a functional gene product. A gene product is functional only if it fulfills its normal (wild-type) functions. Materials and methods of genetically modifying animals are further detailed in U.S. Ser. No. 13/404,662 filed Feb. 24, 2012, Ser. No. 13/467,588 filed May 9, 2012, and Ser. No. 12/622,886 filed Nov. 10, 2009 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm-mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygousity would normally be required. If a particular gene is inactivated by an RNA interference or dominant negative strategy, then heterozygosity is often adequate.

Typically, in embryo/zygote microinjection, a nucleic acid construct or mRNA is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×10⁵ sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs or mRNA can be injected into one of the pronuclei or into the cytoplasm. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al. (1998) Science 280, 1256-1258 and U.S. Pat. No. 6,548,741. For pigs, recipient females can be checked for pregnancy approximately 20-21 days after transfer of the embryos.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animal have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., 1989, Molecular Cloning, A Laboratory Manual, second edition, Cold Spring Harbor Press, Plainview; N.Y. Polymerase chain reaction (PCR) techniques also can be used in the initial screening PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis (1992) Genetic Engineering News 12,1; Guatelli et al. (1990) Proc. Natl. Acad. Sci. USA 87:1874; and Weiss (1991) Science 254:1292. At the blastocyst stage, embryos can be individually processed for analysis by PCR, Southern hybridization and splinkerette PCR (see, e.g., Dupuy et al. Proc Natl Acad Sci USA (2002) 99:4495).

Expression of a nucleic acid sequence encoding a polypeptide in the tissues of transgenic pigs can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of inactivating a gene in a genetically modified animal comprises inducing RNA interference against a target gene and/or nucleic acid such that expression of the target gene and/or nucleic acid is reduced.

For example the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al. (1998) Nature 391:806; Romano and Masino (1992) Mol. Microbiol. 6:3343; Cogoni et al. (1996) EMBO J. 15:3153; Cogoni and Masino (1999) Nature 399:166; Misquitta and Paterson (1999) Proc. Natl. Acad. Sci. USA 96:1451; and Kennerdell and Carthew (1998) Cell 95:1017. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically modified animal such as a livestock animal that express an RNAi directed against a neuroendocrine gene selective for sexual maturation. An embodiment is an RNAi directed against a gene in the group consisting of Gpr54, Kiss1, and GnRH1. The RNAi may be, for instance, selected from the group consisting of siRNA, shRNA, dsRNA, RISC and miRNA.

### Inducible Systems

An inducible system may be used to control expression of a sexual maturation gene. Various inducible systems are known that allow spatiotemporal control of expression of a gene. Several have been proven to be functional in vivo in transgenic animals.

An example of an inducible system is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed foxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a foxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically modified animal such as a livestock animal that comprise a neuroendocrine gene selective for sexual maturation that is under control of an inducible system. The genetic modification of an animal may be genomic or mosaic. An embodiment is a gene in the group consisting of Gpr54, Kiss1, and GnRH1 that is under control of an inducible system. The inducible system may be, for instance, selected from the group consisting of Tet-On, Tet-Off, Cre-lox, and Hif1 alpha.

### Dominant Negatives

Genes may thus be inactivated not only by removal or RNAi suppression but also by creation of a dominant negative phenotype. A dominant negative version of a gene product lacks one or more functions of the wild-type phenotype and dominantly interferes with the function of a normal gene product expressed in the same cell, with a result that the dominant negative phenotype effectively decreases or inactivates the physiological outcome normally expected to be elicited by a gene's normal function. For example, the function of most proteins requires their interaction with other proteins. Such interactions are often required for proper protein localization, ligand binding, protein activation, or the downstream transduction of upstream signals. The mutation of one or more of the components of a multi-protein complex can interfere with one these processes. Thus, the expression of a mutant form of a protein can interfere with a proteins function, even in the presence of a normal gene product, acting as a poison “pill” or a “monkey wrench” into the gearbox. GPCRs are seven-transmembrane (7TM) domain receptors which are trafficked through the biosynthetic pathway to the cell surface in a tightly regulated mechanism with multiple steps and a stringent quality control system to ensure correct GPCR folding and targeting. Association of GPCRs with accessory proteins or chaperones are a key step for the forward trafficking through the endoplasmic reticulum (ER) and Golgi. The life of GPCRs begins in the ER where they are synthesized, folded and assembled. During their migration to the cell surface, GPCRs undergo post-translational modifications to attain mature status. Because the ER forms part of the cellular quality control machinery where functionally inactive mutant GPCRs can be prevented from expression at the cell surface.

Conditions such as X-linked nephrogenic-diabetes insipidus, familial hypocalciuric hypercalcemia, familial glucocorticoid deficiency or hypogonadodotropic hypogonadism are associated with mutations in GPCRs which result in intracellular retention in the ER or Golgi compartments. In numerous cases the defect in cell surface membrane expression is due to intracellular association of receptors, with a dominant-negative (DN) effect of the misfolded receptor on its wild-type counterpart; this DN effect may limit, or even abrogate, plasma membrane expression of the normal receptor and thus provoke a loss-of-function disease (Ulloa-Aguirre et al., 2004a).

Loss-of-function mutations in the GnRHR can lead to partial or complete hypogonadotropic hypogonadism (HH), a failure of pituitary gonadotropes to respond to GnRH, which results in decreased or apulsatile gonadotropin release and reproductive failure. A large number of mutations leading to receptor misfolding and resultant misrouting of the gonadotropin hormone-releasing hormone receptor (GnRHR) in patients with HH have been described (Janovick et al., 2002; Leaños-Miranda et al., 2002; Ulloa-Aguirre et al., 2004b). Many of these mutations act as Dominant negatives for GnRHR function (Pask A J et al, 2005 Mol Endocrinol; Brothers S P et al, 2004 Mol Endocrinol; Leaños-Miranda A et al, 2003 J Clin Endocrinol Metab). Thus, purposeful expression of a DN GnRHR gene is expected to cause sterility in transgenic animals.

As discussed GPR54 is a gatekeeper of the reproductive cascade that initiates puberty. Myriad animal studies have demonstrated that engagement of GPR54 by endogenous peptide ligands, termed kisspeptins, potently stimulates gonadotropin-releasing hormone release from hypothalamic neurons to activate the hypothalamic-pituitary-gonadal axis. Furthermore, the characterization of GPR54 KO mice, which phenocopy the human condition of idiopathic hypogonadotropic hypogonadism, confirmed the essential role of GPR54 for reproductive function. GPCRs are now recognized to exist as multiprotein complexes composed of GPCR-interacting proteins (GIPs) that impart precise spatial and temporal regulation of expression, trafficking, ligand binding, and signaling. GPR54 has been determined to specifically interact with these GIPs. Because the majority of truncated GPCR splice variants act as dominant-negative mutations (Wise 2012, J Mol Signal), the expression of GPR54 lacking one or more transmembrane domains is expected to disrupt the processing/trafficking of endogenous GPR54, thus interfering with its function. Thus, purposeful expression of a DN GPR54 gene is expected to cause sterility in transgenic animals.

### Founder Animals, Animal Lines, Traits, and Reproduction

Founder animals may be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that all of the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Science, 40 (1994) 123-137, U.S. Pat. No. 7,709,206, US 2001/0016315, US 2011/0023140, and US 2005/0153317. An animal line may include a trait chosen from a trait in the group consisting of a production trait, a type trait, a workability trait, a fertility trait, a mothering trait, and a disease resistance trait. Further traits include expression of a recombinant gene product.

Animals with a desired trait or traits may be modified to prevent their sexual maturation. Since the animals are sterile until matured, it is possible to regulate sexual maturity as a means of controlling dissemination of the animals. Animals that have been bred or modified to have one or more traits can thus be provided to recipients with a reduced risk that the recipients will breed the animals and appropriate the value of the traits to themselves. Embodiments of the invention include genetically modifying a genome of an animal with the modification comprising an inactivated sexual maturation gene, wherein the sexual maturation gene in a wild type animal expresses a factor selective for sexual maturation. Embodiments include treating the animal by administering a compound to remedy a deficiency caused by the loss of expression of the gene to induce sexual maturation in the animal.

Breeding of animals that require administration of a compound to induce sexual maturity may advantageously be accomplished at a treatment facility. The treatment facility can implement standardized protocols on well-controlled stock to efficiently produce consistent animals. The animal progeny may be distributed to a plurality of locations to be raised. Farms and farmers (a term including a ranch and ranchers) may thus order a desired number of progeny with a specified range of ages and/or weights and/or traits and have them delivered at a desired time and/or location. The recipients, e.g., farmers, may then raise the animals and deliver them to market as they desire.

Embodiments include delivering (e.g., to one or more locations, to a plurality of farms) a genetically modified livestock animal having an inactivated neuroendocrine gene selective for sexual maturation. Embodiments include delivery of animals having an age of between about 1 day and about 180 days. The animal may have one or more traits (for example one that expresses a desired trait or a high-value trait or a novel trait or a recombinant trait). Embodiments further include providing said animal and/or breeding said animal.

### Recombinases

Embodiments of the invention include administration of a TALEN or TALENs or a Zinc finger nuclease with a recombinase or other DNA-binding protein associated with DNA recombination. A recombinase forms a filament with a nucleic acid fragment and, in effect, searches cellular DNA to find a DNA sequence substantially homologous to the sequence. An embodiment of a TALEN-recombinase embodiment comprises combining a recombinase with a nucleic acid sequence that serves as a template for HDR. The HDR template sequence has substantial homology to a site that is targeted for cutting by the TALEN/TALEN pair. As described herein, the HDR template provides for a change to the native DNA, by placement of an allele, creation of an indel, insertion of exogenous DNA, or with other changes. The TALEN is placed in the cell or embryo by methods described herein as a protein, mRNA, or by use of a vector. The recombinase is combined with the HDR template to form a filament and placed into the cell. The recombinase and/or HDR template that combines with the recombinase may be placed in the cell or embryo as a protein, an mRNA, or with a vector that encodes the recombinase. The disclosure of US Pub 2011/0059160 (U.S. Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assist in repair of DNA double strand breaks. RAD51 family members are homologous to the bacterial RecA and yeast Rad51 genes. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

RecA is known for its recombinase activity to catalyze strand exchange during the repair of double-strand breaks by homologous recombination (McGrew, 2003) Radding, et al., 1981; Seitz et al., 1998). RecA has also been shown to catalyze proteolysis, e.g., of the LexA and repressor proteins, and to possess DNA-dependent ATPase activity. After a double-strand break occurs from ionizing radiation or some other insult, exonucleases chew back the DNA ends 5′ to 3′, thereby exposing one strand of the DNA (McGrew, 2003; Cox, 1999). The single-stranded DNA becomes stabilized by single-strand binding protein (SSB). After binding of SSB, RecA binds the single-stranded (ss) DNA and forms a helical nucleoprotein filament (referred to as a filament or a presynaptic filament). During DNA repair, the homology-searching functions of RecA direct the filament to homologous DNA and catalyze homologous base pairing and strand exchange. This results in the formation of DNA heteroduplex. After strand invasion, DNA polymerase elongates the ssDNA based on the homologous DNA template to repair the DNA break, and crossover structures or Holliday junctions are formed. RecA also shows a motor function that participates in the migration of the crossover structures (Campbell and Davis, 1999).

Recombinase activity comprises a number of different functions. For example, polypeptide sequences having recombinase activity are able to bind in a non-sequence-specific fashion to single-stranded DNA to form a nucleoprotein filament. Such recombinase-bound nucleoprotein filaments are able to interact in a non-sequence-specific manner with a double-stranded DNA molecule, search for sequences in the double-stranded molecule that are homologous to sequences in the filament, and, when such sequences are found, displace one of the strands of the double-stranded molecule to allow base-pairing between sequences in the filament and complementary sequences in one of the strands of the double stranded molecule. Such steps are collectively denoted “synapsis.”

RecA and RecA-like proteins (called Rad51 in many eukaryotic species) have been examined for stimulating gene targeting and homologous recombination in a variety of eukaryotic systems. In tobacco cells, expression of bacterial RecA containing a nuclear localization signal (NLS) increases the repair of mitomycin C-induced DNA damage by homologous recombination and somatic intrachromosomal recombination (recombination between homologous chromosomes) from three to ten fold (Reiss, 1996). Expression of NLSRecA in tobacco can also stimulate sister chromatid exchange 2.4-fold over wild-type levels (Reiss, 2000). In somatic mammalian cells, overexpression of NLSRecA stimulates gene-targeting by homologous recombination 10-fold (Shcherbakova, 2000). However, in human cells, overexpression of a human homologue of RecA, hRAD51, only stimulates recombination 2 to 3-fold over wild type levels under the antibiotic selection (Yanez, 1999). In zebrafish, a mutant form of the enhanced green fluorescent protein (EGFP) was corrected at low frequency by injecting ssDNA-RecA filaments directly (Cui, 2003). Rad52, a member of the Rad51 epistasis group, also promotes single-strand annealing and low level gene inactivation in zebrafish using mutated oligonucleotides (Takahashi, 2005). Taken together, these studies indicate that ectopic expression of RecA or Rad51 results in a modest stimulation of homologous recombination but does not increase levels sufficiently to be useful for gene-targeting.

Thus recombinase activities include, but are not limited to, single-stranded DNA-binding, synapsis, homology searching, duplex invasion by single-stranded DNA, heteroduplex formation, ATP hydrolysis and proteolysis. The prototypical recombinase is the RecA protein from E. coli. See, for example, U.S. Pat. No. 4,888,274. Prokaryotic RecA-like proteins have also been described in Salmonella, Bacillus and Proteus species. A thermostable RecA protein, from Thermus aquaticus, has been described in U.S. Pat. No. 5,510,473. A bacteriophage T4 homologue of RecA, the UvsX protein, has been described. RecA mutants, having altered recombinase activities, have been described, for example, in U.S. Pat. Nos. 6,774,213; 7,176,007 and 7,294,494. Plant RecA homologues are described in, for example, U.S. Pat. Nos. 5,674,992; 6,388,169 and 6,809,183. RecA fragments containing recombinase activity have been described, for example, in U.S. Pat. No. 5,731,411. Mutant RecA proteins having enhanced recombinase activity such as, for example, RecA803 have been described. See, for example, Madiraju et al. (1988) Proc. Natl. Acad. Sci. USA 85:6592-6596.

A eukaryotic homologue of RecA, also possessing recombinase activity, is the Rad51 protein, first identified in the yeast Saccharomyces cerevisiae. See Bishop et al., (1992) Cell 69: 439-56 and Shinohara et al, (1992) Cell: 457-70 Aboussekhra, et al., (1992) Mol. Cell. Biol. 72, 3224-3234. Basile et al., (1992) Mol. Cell. Biol. 12, 3235-3246. Plant Rad51 sequences are described in U.S. Pat. Nos. 6,541,684; 6,720,478; 6,905,857 and 7,034,117. Another yeast protein that is homologous to RecA is the Dmc1 protein. RecA/Rad51 homologues in organisms other than E. coli and S. cerevisiae have been described. Morita et al. (1993) Proc. Natl. Acad. Sci. USA 90:6577-6580; Shinohara et al. (1993) Nature Genet. 4:239-243; Heyer (1994) Experientia 50:223-233; Maeshima et al. (1995) Gene 160:195-200; U.S. Pat. Nos. 6,541,684 and 6,905,857.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

Additional descriptions of proteins having recombinase activity are found, for example, in Fugisawa et al. (1985) Nuc. Acids Res. 13:7473; Hsieh et al. (1986) Cell 44:885; Hsieh et al. (1989) J. Biol. Chem. 264:5089; Fishel et al. (1988) Proc. Natl. Acad. Sci. USA 85:3683; Cassuto et al. (1987) Mol. Gen. Genet. 208:10; Ganea et al. (1987) Mol. Cell Biol. 7:3124; Moore et al. (1990) J. Biol. Chem.: 11108; Keene et al. (1984) Nucl. Acids Res. 12:3057; Kimiec (1984) Cold Spring Harbor Symp. 48:675; Kimeic (1986) Cell 44:545; Kolodner et al. (1987) Proc. Natl. Acad. Sci. USA 84:5560; Sugino et al. (1985) Proc. Natl. Acad, Sci. USA 85: 3683; Halbrook et al. (1989) J. Biol. Chem. 264:21403; Eisen et al. (1988) Proc. Natl. Acad Sci. USA 85:7481; McCarthy et al. (1988) Proc. Natl. Acad. Sci. USA 85:5854; and Lowenhaupt et al. (1989) J. Biol. Chem. 264:20568, which are incorporated herein by reference. See also Brendel et al. (1997) J. Mol. Evol. 44:528.

Examples of proteins having recombinase activity include recA, recA803, uvsX, and other recA mutants and recA-like recombinases (Roca (1990) Crit. Rev. Biochem. Molec. Biol. 25:415), (Kolodner et al. (1987) Proc. Natl. Acad Sci. U.S.A. 84:5560; Tishkoff et al. (1991) Molec. Cell. Biol. 11:2593), RuvC (Dunderdale et al. (1991) Nature 354:506), DST2, KEM1 and XRN1 (Dykstra et al. (1991) Molec. Cell. Biol. 11:2583), STPa/DST1 (Clark et al. (1991) Molec. Cell. Biol. 11:2576), HPP-1 (Moore et al. (1991) Proc. Natl. Acad. Sci. U.S.A. 88:9067), other eukaryotic recombinases (Bishop et al (1992) Cell 69:439; and Shinohara et al. (1992) Cell 69:457); incorporated herein by reference.

In vitro-evolved proteins having recombinase activity have been described in U.S. Pat. No. 6,686,515. Further publications relating to recombinases include, for example, U.S. Pat. Nos. 7,732,585, 7,361,641, 7,144,734. For a review of recombinases, see Cox (2001) Proc. Natl. Acad Sci. USA 98:8173-8180.

A nucleoprotein filament, or “filament” may be formed. The term filament, in the context of forming a structure with a recombinase, is a term known to artisans in these fields. The nucleoprotein filament so formed can then be, e.g., contacted with another nucleic acid or introduced into a cell. Methods for forming nucleoprotein filaments, wherein the filaments comprise polypeptide sequences having recombinase activity and a nucleic acid, are well-known in the art. See, e.g., Cui et al. (2003) Marine Biotechnol. 5:174-184 and U.S. Pat. Nos. 4,888,274; 5,763,240; 5,948,653 and 7,199,281, the disclosures of which are incorporated by reference for the purposes of disclosing exemplary techniques for binding recombinases to nucleic acids to form nucleoprotein filaments.

In general, a molecule having recombinase activity is contacted with a linear, single-stranded nucleic acid. The linear, single-stranded nucleic acid may be a probe. The methods of preparation of such single stranded nucleic acids are known. The reaction mixture typically contains a magnesium ion. Optionally, the reaction mixture is buffered and optionally also contains ATP, dATP or a nonhydrolyzable ATP analogue, such as, for example, γ-thio-ATP (ATP-γ-S) or γ-thio-GTP (GTP-γ-S). Reaction mixtures can also optionally contain an ATP-generating system. Double-stranded DNA molecules can be denatured (e.g., by heat or alkali) either prior to, or during, filament formation. Optimization of the molar ratio of recombinase to nucleic acid is within the skill of the art. For example, a series of different concentrations of recombinase can be added to a constant amount of nucleic acid, and filament formation assayed by mobility in an agarose or acrylamide gel. Because bound protein retards the electrophoretic mobility of a polynucleotide, filament formation is evidenced by retarded mobility of the nucleic acid. Either maximum degree of retardation, or maximum amount of nucleic acid migrating with a retarded mobility, can be used to indicate optimal recombinase:nucleic acid ratios. Protein-DNA association can also be quantitated by measuring the ability of a polynucleotide to bind to nitrocellulose.

Patent applications, patents, publications, and journal articles set forth herein are hereby incorporated herein by reference for all purposes; in case of conflict, the specification is controlling.

### EXAMPLES

General techniques including making of TALENs, are generally described in US2013/0117870 unless otherwise indicated. Some of these general techniques are further described herein. And the certain of the Examples provide detailed experimental data and results.

### Example 1

TALEN designing and production; general conditions. Candidate TALEN target DNA sequences and RVD sequences were identified using the online tool “TAL EFFECTOR NUCLEOTIDE TARGETER”. Plasmids for TALEN DNA transfection or in vitro TALEN mRNA transcription were then constructed by following the Golden Gate Assembly protocol using pCGOLDYTALEN (Addgene ID 38143) and RCIscript-GOLDYTALEN (Addgene ID 38143) as final destination vectors (Carlson 2012). The final pC-GoldyTALEN vectors were prepared by using PureLink® HIPURE PLASMID MIDIPREP Kit (Life Technologies) and sequenced before usage. Assembled RCIscript vectors prepared using the QIAPREP SPIN MINIPREP kit (Qiagen) were linearized by SacI to be used as templates for in vitro TALEN mRNA transcription using the mMESSAGE mMACHINE® T3 Kit (Ambion) as indicated previously. Modified mRNA was synthesized from RCIScript-GOLDYTALEN vectors as previously described Carlson 2012) substituting a ribonucleotide cocktail consisting of 3′-0-Mem7G(5′)ppp(5′)G RNA cap analog (New England Biolabs), 5-methylcytidine triphosphate pseudouridine triphosphate (TriLink Biotechnologies, San Diego, Calif.) and adenosine triphosphate guanosine triphosphate. Final nucleotide reaction concentrations are 6 mM for the cap analog, 1.5 mM for guanosine triphosphate, and 7.5 mM for the other nucleotides. Resulting mRNA was DNAse treated prior to purification using the MEGACLEAR REACTION CLEANUP kit (Applied Biosciences).

Tissue culture and transfection; general conditions. Pig or cattle fibroblasts were maintained at 37 or 30° C. (as indicated) at 5% CO2 in DMEM supplemented with 10% fetal bovine serum, 100 I.U./ml penicillin and streptomycin, and 2 mM L-Glutamine. For transfection, all TALENs and HDR templates were delivered through transfection using the Neon Transfection system (Life Technologies) unless otherwise stated. Briefly, low passage Ossabaw, Landrace, Wagyu, or Holstein fibroblasts reaching 100% confluence were split 1:2 and harvested the next day at 70-80% confluence. Each transfection was comprised of 500,000-600,000 cells resuspended in buffer “R” mixed with plasmid DNA or mRNA and oligos and electroporated using the 100 μl tips by the following parameters: input Voltage; 1800V; Pulse Width; 20 ms; and Pulse Number; 1. Typically, 2-4 μg of TALEN expression plasmid or 1-2 μg of TALEN mRNA and 2-3 μM of oligos specific for the gene of interest were included in each transfection. Deviation from those amounts is indicated in the figure legends. After transfection, cells were divided 60:40 into two separate wells of a 6-well dish for three days' culture at either 30 or 37° C. respectively. After three days, cell populations were expanded and at 37° C. until at least day 10 to assess stability of edits.

### Example 2

### Dilution Cloning

Dilution cloning was used in some cases, as indicated. Three days post transfection, 50 to 250 cells were seeded onto 10 cm dishes and cultured until individual colonies reached about 5 mm in diameter. At this point, 6 ml of TrypLE (Life Technologies) 1:5 (vol/vol) diluted in PBS was added and colonies were aspirated, transferred into wells of a 24-well dish well and cultured under the same conditions. Colonies reaching confluence were collected and divided for cryopreservation and genotyping. Sample preparation: Transfected cells populations at day 3 and 10 were collected from a well of a 6-well dish and 10-30% were resuspended in 50 of 1× PCR compatible lysis buffer: 10 mM Tris-Cl pH 8.0, 2 mM EDTA, 0.45% Tryton X-100 (vol/vol), 0.45% Tween-20 (vol/vol) freshly supplemented with 200 μg/ml Proteinase K. The lysates were processed in a thermal cycler using the following program: 55° C. for 60 minutes, 95° C. for 15 minutes. Colony samples from dilution cloning were treated as above using 20-30 μl of lysis buffer.

### Example 3

### Mutation Detection and RFLP Analysis

PCR flanking the intended sites was conducted using PLATINUM TAQ DNA POLYMERASE HIFI (Life Technologies) with 1 μl of the cell lysate according to the manufacturer's recommendations. The frequency of mutation in a population was analyzed with the SURVEYOR MUTATION DETECTION Kit (Transgenomic) according to the manufacturer's recommendations using 10 ul of the PCR product as described above. RFLP analysis was performed on 10 μl of the above PCR reaction using the indicated restriction enzyme. SURVEYOR and RFLP reactions were resolved on a 10% TBE polyacrylamide gels and visualized by ethidium bromide staining. Densitometry measurements of the bands were performed using ImageJ; and mutation rate of SURVEYOR reactions was calculated as described in Guschin et al. 2010. Percent HDR was calculated via dividing the sum intensity of RFLP fragments by the sum intensity of the parental band+RFLP fragments. For analysis of mloxP insertion, small PCR products spanning the insertion site were resolved on 10% polyacrylamide gels and the insert versus wild type alleles could be distinguished by size and quantified. RFLP analysis of colonies was treated similarly except that the PCR products were amplified by 1× MYTAQ RED Mix (Bioline) and resolved on 2.5% agarose gels. For analysis of clones for introgression of the GDF8 G938A-only (oligos lacked a novel RFLP), colonies were initially screened by a three primer assay that could distinguish between heterozygous ad homozygous introgression; Briefly, lysates from pig or cattle colonies were analysed by PCR using 1× MYTAQ RED MIX (Bioline) using the following primers and programs. Cattle GDF8 (Outside F1: 5′-CCTTGAGGTAGGAGAGTGTTTTGGG (SEQ ID NO: 3), Outside R1: 5′-TTCACCAGAAGACAAGGAGAATTGC (SEQ ID NO: 1), Inside F1: 5′-TAAGGCCAATTACTGCTCTGGAGACTA (SEQ ID NO: 2); and 35 cycles of (95° C., 20 s; 62° C., 20 s; 72° C., 60 s). Pig GDF8: Outside F1: 5′-CCTTTTTAGAAGTCAAGGTAACAGACAC (SEQ ID NO: 4), Outside R1: 5′-TTGATTGGAGACATCTTTGTGGGAG (SEQ ID NO: 5), Inside F1: 5′-TAAGGCCAATTACTGCTCTGGAGATTA (SEQ ID NO: 6); and 35 cycles of (95° C., 20 s; 58° C., 20 s; 72° C., 60 s) Amplicons from candidates were sequenced directly and/or TOPO cloned (Life Technologies) and sequenced by Sanger sequencing. To detect TALEN-mediated HDR at with the BB-HDR template, either 1 μl or 1 μl of a 1:10 dilution of PCR-lysate (1,000 cells/ul) was added to a PCR reaction with PCR primers bt GDF8 BB 5-1 (primer “c”) and primer “c” (BB-Detect 3-1-5′-GCATCGAGATTCTGTCACAATCAA (SEQ ID NO: 7)) and subjected to PCR with using 1× MYTAQ RED MIX (Bioline) for 40 cycles (9 459 5° C., 20 s; 66° C., 20 s; 72° C., 60 s). To confirm HDR in colonies identified by the above PCR, amplification of the entire locus was performed with primers bt GDF8 BB 5-1 and bt GDF8 BB 3-1 followed by TOPO cloning (Life Technologies) and sequencing.

### Example 4

### Confirmation of Belgian Blue Introgression By Sequencing

The schematics of Wagyu wild-type GDF8 and the Belgian Blue template (BB-HDR) are shown in FIG. 2. PCR was conducted using primers located outside of the homology arms (c and d) on five PCR positive colonies followed by cloning and sequencing with primer b′. Comparison to the wild-type sequence reveals the expected 11-basepair deletion characteristic the Belgian Blue allele (heterozygous) in 4 of 5 colonies. TALENs (btGDF83.1) and a dsDNA template (BB-HDR) were designed to introduce an 11-basepair deletion into exon-3 of bovine GDF8 (Belgium Blue mutation) by Double-Strand Break-induced homologous recombination. Half of the binding site for the left TALEN is missing in the BB-HDR template and thus should be resistant to TALEN cleavage. SURVEYOR assay demonstrated activity of btGDF83.1 TALENs at both 37 and 30° Celsius. Allele-specific PCR demonstrated that HDR induction was dependent on co-transfection of TALENs and the BB-HDR template. The PCR assay was developed to specifically detect HDR modified GDF8 alleles using primers c and c′. The 3′ end of primer c′ spans the 11-basepair deletion, and cannot amplify the wild type allele (wt). Five hundred cell equivalents were included in each PCR reaction including the positive control. Percent HDR was determined by comparative densitometry between experimental and control reactions.

### Example 5

### Precision Alteration of Intended Gene in Wild-Type Wagyu Cattle

A gene of wild-type Wagyu cattle was altered by making a deletion in a targeted area of the gene (an 11 bp deletion). This alteration made the Wagyu cattle have the allele of Beligan Blue cattle. When transfected alone, the btGDF8.1 TALEN pair cleaved up to 16% of chromosomes at the target locus. TALENs (btGDF83.1) and a dsDNA template (BB-HDR) were designed to introduce an 11-bp deletion in exon-3 of bovine GDF8 (Belgium Blue mutation) by DSB induced homologous recombination. Half of the binding site for the left TALEN was missing in the BB-HDR template, to make it resistant to TALEN cleavage. A SURVEYOR assay demonstrated activity of btGDF83.1 TALENs at both 37 and 30° Celsius. The PCR product used for this assay was generated using primers b and b′ (not shown). The BB-HDR template was not included in these replicates since it would confound estimates of btGDF83.1 activity. Allele specific PCR demonstrated that HDR induction was dependent on co-transfection of TALENs and the BB-HDR template. The PCR assay was developed to specifically detect HDR modified GDF8 alleles using primers c and c′ (not shown). The 3′ end of primer c′ spanned the 11-bp deletion so that it could not amplify the wild type allele “wt”. Five hundred cell equivalents were included in each PCR reaction including the positive control “C”. Percent HDR was determined by comparative densitometry between experimental and control reactions. Co-transfection with a supercoiled DNA template harboring a 1623 bp DNA fragment from Belgian Blue cattle resulted in a gene conversion frequency (HDR) of 0.5% to 5% as suggested by semi-quantitative PCR at day 3, without selection for the desired event. These results demonstrated that TALENs can be used to effectively place exogenous nucleic acid sequences in livestock, including alleles—and without markers. To assess the frequency of placement in individual colonies, the transposon co-selection strategy was implemented to isolate and expand individual colonies for DNA sequencing. Gene conversion using template from Belgian Blue cattle was detected in 5 colonies out of 366 examined by PCR. Amplification with primers outside the Belgian Blue HDR template and sequencing confirmed the presence of the expected 11 bp deletion in 4 of the colonies.

A second repeat experiment was performed with consistent results, with about 1% of all tested colonies being positive for bi-allelic conversion and about 0.5% to about 1% of all tested colonies being heterozygous for allele conversion.

Similarly, alleles were also introduced into pig (Ossabaw) cells using oligo HDR The cells were modified with a combination of mRNA encoded TALENs and single-stranded oligonucleotides to place an allele that occurs naturally in one species to another species (interspecific migration). Piedmontese GDF8 SNP C313Y, were chosen as an example and was introduced into Ossabow swine cells. No markers were used in these cells at any stage. A similar peak in HDR was observed between pig and cattle cells at 0.4 nmol ssODN, (not shown) however, HDR was not extinguished by higher concentrations of ssODN in Ossabaw fibroblasts.

### Example 6

### Modification at Intended Targets

Consistent modification of targeted genes was made. Referring to FIG. 4, each chart displays results of targeting a specific locus in fibroblasts (e.g., ssIL2RG; “ss” for Sus scrofa and “bt” for Bos taurus) using oligo donor templates and TALENs delivered as plasmid DNA or mRNA. (Insets) Diagrams of the oligo templates, in which the shaded boxes represent the TALEN-binding site and the spacers are shown in white. Each oligo contains either a 4-bp insertion (ins4) or deletion (del4) that introduces a novel restriction site for RFLP analysis. Presumptive blocking mutations (BM) replace the conserved −1 thymidine (relative to the TALEN-binding site) with the indicated nucleotide. Fibroblasts were transfected with either TALEN-encoding plasmids (3 μg) or mRNA (1 μg) along with 3 μM of their cognate oligo-homologous template. Cells were then incubated at 37° C. or 30° C. for 3 d before expansion at 37° C. until day 10. TALEN activity was measured by the Surveyor assay at day 3 (Day3 Surveyor), and HDR was measured at days 3 and 10 by RFLP analysis (Day 3 % HDR and Day 10 % HDR). Each bar displays the average and SEM from three replicates. Each of the targeted loci was successfully modified.

### Example 7

### High Efficiency for Making Intended Changes in Genes

FIG. 5 shows analysis of changes made to genes APC, LDLR, p53, p65, and btGDF8. In some cases insertions were intended, while SNPs were intended in other cases. Changes were made with TALENs and HDR templates, as described above. The count of perfect, intended HR reads versus the wild type reads is plotted for: insertion (panel a) and SNP alleles (panel b). Sequence analysis of TALEN stimulated HDR alleles was made. PCR amplicons flanking the target site (200-250 bp total) derived from TALEN mRNA and oligo transfected cell populations were sequenced by ILLUMINA sequencing. Total read count ranged from 10,000 to 400,000 per sample. The target locus, time point and whether or not BMs were included in the oligo are indicated below. Panel c shows reads from btGDF8 and p65, as sorted for incorporation of the target SNP and then classified intended (iSNP) versus those with an additional mutation (iSNP+Mut) and plotted against the total number of reads. Accordingly, in the case where only a single SNP was intended, there were also additional changes, as indicated.

### Example 8

### Frequencies for Recovery of Colonies with HDR Alleles

Table 1, entitled Frequencies for recovery of colonies with HDR alleles, lists the results of an analysis of about 650 colonies of cells for intended indel alleles in eight separate loci. The analysis revealed a recovery rate of 10-64% (average, 45%), with up to 32% of the colonies homozygous for the edit. Changes were made with TALENs and HDR templates, as described above. The colonies were obtained by dilution cloning without drug selection.

### Example 9

### Cloned Pigs with HDR Alleles of DAZL and APC

FIG. 6 shows a genetic analysis of cloned animals. Two gene-edited loci in the porcine genome, deleted in azoospermia-like (DAZL) and adenomatous polyposis coli (APC) were chosen. Colonies of cultured cells treated with HDR- or NHEJ edited alleles of DAZL or APC were pooled for cloning by chromatin transfer (CT). Each pool yielded two pregnancies from three transfers, of which one pregnancy each was carried to term. A total of eight piglets were born from DAZL-modified cells, each of which reflected genotypes of the chosen colonies consistent with either the HDR allele (founders 1650, 1651, and 1657) or deletions resulting from NHEJ (FIG. 5A). Three of the DAZL piglets (founders 1655-1657) were stillborn. Of the six piglets from APC-modified cells, one was stillborn, three died within 1 wk, and another died after 3 wk, leaving only founder 1661 alive. The lack of correlation between genotype and survival suggests that the early deaths were related to cloning rather than to gene edits. All six APC piglets were heterozygous for the intended HDR-edited allele, and all but one piglet had either an in-frame insertion or deletion of 3 by on the second allele (FIGS. 6a and b). The remaining animals are being raised for phenotypic analyses of spermatogenesis arrest (DAZL−/− founders) or development of colon cancer (APC+/− founders). Referring to FIG. 6, (a) RFLP analysis of cloned piglets derived from DAZL- and APC-modified landrace and Ossabaw fibroblasts, respectively. Expected RFLP products for DAZL founders are 312, 242, and 70 bp (open triangles), and those for APC are 310, 221, and 89 bp (filled triangles). The difference in size of the 312-bp band between WT and DAZL founders reflects the expected deletion alleles. (b) Sequence analysis confirming the presence of the HDR allele in three of eight DAZL founders, and in six of six APC founders. BMs in the donor templates (HDR) are indicated with arrows, and inserted bases are enclosed in blocks. The bold text in the top WT sequence indicates the TALEN-binding sites.

### Example 10

### GPR54 Knockout

FIG. 7 depicts GPR54 knockouts, made according to the indicated gene targeting strategy. TALENs designed to bind porcine exon 3 (underlined text) were co-transfected with an oligonucleotide homology template (HDR) designed to introduce a premature stop codon (boxed) and a HindIII restriction site. For the experimental results shown in panel b, 2 micrograms of TALENs encoding mRNA plus 0.2 nMol (2 uM) of the HDR template were transfected into pig fibroblasts 500,000 pig fibroblasts using the NEON nucleofection system (Life Technologies) with the following settings: 1 pulse, 1800 v; 20 ms width and a 100 ul tip. The cells were grown at 30° C. for three days after exposure to TALENs and cells were enumerated and plated at a range of densities 1-20 cells/cm2 on 10 cm dishes. Cells were cultured for 10-15 days until individual colonies of 3-4 mm in diameter could be observed. Colonies were aspirated with a p-200 pipettor under gentle aspiration and expelled into a well of 24-well plate with 500 ul of growth medium (Carlson, 2011). Plates with clearly defined colonies (˜10-30/plate) were chosen for colony aspiration to limit the chance of aspirating cells from multiple colonies. Once a colony reached 70-90 percent confluent in the 24-well dish, a portion was harvested for RFLP analysis and the remainder was cryopreserved. Panel b) A total of 96 colonies were analyzed for homology dependent repair by HindIII RFLP assay. DNA from each colony was added to a PCR reaction that included PCR primers flanking the target site; forward (5′-aaggatgtcagcacctctctggg (SEQ ID NO: 8)) and reverse (5′-ACCCACCCGGACTCTACTCCTACCA (SEQ ID NO: 9)). PCR products (389 bp) were added to a HindIII restriction digest and resolved on a 2.5% agarose gel. Each lane represents one colony. Cleavage products of 231 and 158 bp are indicative of homology dependent repair. Colonies with the parent band of 389 bp are classified as heterozygous (open triangle) and those without are classified as homozygous (filled triangle) for the HDR, knockout allele. The cells, or cells prepared by this technique, will be used to clone animals using customary techniques.

### Example 11

### Creation of Livestock That Do Not Mature Without Treatment

Livestock with GPR54 knockout(s) will be prepared, including cattle, pig, and chicken. The preceding example details one such process. The following specific methods are described for pigs; artisans will be able to adapt the experiments to other livestock after reading this application. TALENs for Gpr54 will be developed and used to generate heterozygous and homozygous knockout cell lines. Pregnancy will be established using male and female Gpr54^{-/-} and/or cell lines heterozygous for Gpr54^{+/-} with Gpr54^{-/-} animals generated by intercross. The development and fertility of Gpr54^{-/-} animals will be evaluated. After establishing that Gpr54^{-/-} animals do not progress to puberty and are indeed sterile, experiments will be performed for restoring fertility by gonadotropin or GnRH1 treatment. The already-demonstrated ability to generate efficient TALENs, isolate mutant colonies and produce transgenic animals from cells or zygotes has been well documented herein, see also Tan et al., PNAS, 110(41): 16526-16531, 2013.

Generation of Gpr54^{-/-} male and female pigs. Ten bi-allelic KO male and female clones, as generated in Example 11, harboring frame shift mutations of both alleles will be pooled for cloning by SCNT. Two rounds of cloning (3 transfers each) will be conducted. If no pregnancies are established in the first round with Gpr54^{-/-}, round-2 cloning will be conducted with Gpr54^{+/-} cells. Genotypes of the resulting animals will be characterized by sequencing of the targeted region of Gpr54. If Gpr54^{+/-} cells were used for cloning, Gpr54^{-/-} animals will be generated by intercross.

Phenotypic evaluation of Gpr54^{-/-} pigs. Serum levels of testosterone and FSH (≧3 per sex) will be quantified every two weeks for Gpr54^{-/-} animals and age-matched controls beginning at 5 months and continuing to 9 months of age. For males, testicular size will be measured and plotted against body weight and age. At 9 months of age, if Gpr54^{-/-} swine deviate from wild type control animals and fail to show serology or behavior traits (i.e., mounting) indicative of puberty, at least one male and female will be sacrificed for anatomical and histological assessment of reproductive organs by qualified a pathologists.

Evaluation of GnRH1 injection as a means to restore fertility in Gpr54^{-/-} pigs. Gonadotropin or pulsatile GnRH 1 therapy is an effective treatment for restoring fertility in humans with HH. (Buchter, D., Behre, H. M., Kliesch, S., and Nieschlag, E. (1998). Pulsatile GnRH1 or human chorionic gonadotropin/human menopausal gonadotropin as effective treatment for men with hypogonadotropic hypogonadism: a review of 42 cases. Eur J Endocrinol 139, 298-303. For either therapy to be successful, a positive response in levels of serum LH and testosterone must be apparent. Therefore, we seek to determine whether infertile Gpr54^{-/-} display the typical spike in LH or testosterone in response to GnRH1 injection. (Wise, T., Zanella, E. L., Lunstra, D. D., and Ford, J. J. (2000). Relationships of gonadotropins, testosterone, and cortisol in response to GnRH1 and GnRH1 antagonist in boars selected for high and low follicle-stimulating hormone levels. J Anim Sci 78, 1577-1590.) Jugular catheters will be placed in subject boars (n>3) for repeated blood sampling. Samples will be taken every 20 minutes, and a single bolus of GnRH1 (100 ng/kg body weight) will be administered at 120 minutes. After GnRH1 injection, sampling frequency occur every 10 minutes for 30 minutes to monitor the LH surge, followed by an additional four hours of sampling every 20 minutes.

Gene Inactivation by Dominant Negatives Similar processes may be used to express dominant negatives that inactivate one or more of the genes. And, for instance, transposons encoding a dominant negative may be inserted into a chromosome by a suitable transposase. The treatment to restore fertility may be, e.g., dietary treatment with pharmacoperones for fertility rescue.

### Example 12

CRISPR gRNAs that overlapped the T1591C site of p65 were made and evaluated for introgression. Efficient production of double stranded breaks (DSBs) at the intended site was observed. CRISPR/Cas9-mediated HDR was <6% at day 3 and below the limit of detection at day 10. Recovery of modified clones was lower with CRISPR-mediated HDR than with TALENs, even though the TALENs cut 35 bp away from the SNP site (Table 1). Analysis of CRISPR/Cas9-induced targeting at a second locus, sAPC14.2, was more efficient, although it did not reach the level of HDR induced by TALENs at this site (˜30% vs. 60%). See also, Tan et al., PNAS, 110(41): 16526-16531, 2013). The CRISPR/Cas9 endonucleases were generated based on the Church laboratory system and methods, Mali P, et al. (2013) RNA-guided human genome engineering via Cas9. Science 339(6121):823-826.

### Example 13

Referring to FIG. 8, the structural organization of the kiss gene is conserved and contains two coding exons, one encoding both the signal peptide and part of the kisspeptin precursor, the other encoding the remainder of the precursor including the kisspeptin-10 sequence. The position of the intron on tilapia Kiss mRNA is indicated by a triangle glyph. The location of the forward and reverse primers for PCR amplification of the target region (442 bp) and. The binding sites for the two engineered pairs of TALENs, Kiss1.1a and Kiss1.1b are indicated in black and gray boxes. Panel b shows a schematic representation of the targeted kiss genomic region showing the location of the kisspeptin-10 biologically active peptide and each kiss1.1a and 1b TALENs recognition sites. PCR (442 bp) and qPCR primer pairs (138 bp amplicon) for analysis of indels are shown as well.

### Example 14

### Kiss and KissR Knockout in Fish

### A. Construction of TALEN Expression Vectors

Table showing construction:

| | Sense Left TALEN Sense Spacer Antisense Right TALEN |
|---|---|
| Kiss1.1a | ACAACCCTCTCAGCCTT CGCTTTGGGAAACGCT ACAATGGCTACATTTAC |
| | (SEQ ID NO: 10) |
| Kiss1.1b | CGCTTTGGGAAACGCTACAAT GGCTACATTTACAGA AGAGCTGTTAAAAGAGCC |
| | (SEQ ID NO: 11) |
| KissR E2 | CCCCTTCACCGCCACCCTTT ACCCCCTCCCTGGATGG ATCTTTGGCAACTTCATGTGC |
| | (SEQ ID NO: 12) |
| KissR E3 | CTACCCCCTGAAATCTCTT CGGCACCGAACCCCCA AAGTAGCCATGATTGTCAGC |
| | (SEQ ID NO: 13) |

Table of primers used

| Target site | Primer Name | Primer sequence (5′-3′) | Experiment | |
|---|---|---|---|---|
| KissRE2 | QPCRE2 F | GCCACTGACATCATCTTCTTG | qPCR (112 bp) | SEQ ID NO: 14 |
| | QPCRE2 R2 | GAAACAGAAAGTTGAAGTGG | | SEQ ID NO: 15 |
| KissRE3 | QPCRE3 F | TCACCCTGACTGCTATGAGTGGA | qPCR (143 bp) | SEQ ID NO: 16 |
| | QPCRE3 R2 | ATGAGTCAGTCGATAATGACACG | sequencing | SEQ ID NO: 17 |
| KissRE2 | GKRE2F | TTATGCAAAAGAAGAAAGGTG | PCR (622 bp) | SEQ ID NO: 18 |
| | GKRE2R | GCAGAGTTCGACCTACTTTCATTG | | SEQ ID NO: 19 |
| KissRE3 | GKRE3F | TATACATAGCCCCCATTTTC AGTG | PCR (702 bp) | SEQ ID NO: 20 |
| | GKRE3R | GGCAGCAGGTAGGCAGCAA | | SEQ ID NO: 21 |
| Kiss1.1a | KissF | GTCCTCTGCATTCAGGAGA ACAG | PCR (442 bp) | SEQ ID NO: 22 |
| and b | KissR | CTAAAAGTATTTTATTTACATAGT | | SEQ ID NO: 23 |
| Kiss1.1a | QPCRkissF | AGGCAGCTCCTTTGCAATGAT | qPCR (138 bp) | SEQ ID NO: 24 |
| | QPCRkissR | AGAGAAGGGTGAAAACTTTTT | | SEQ ID NO: 25 |

B. TALEN mRNA Synthesis.

MINIPREP DNA of pT3Ts-TALEN were digested with 5-10× Units of SacI-high fidelity for 2 hours in a 200-μL reaction. Restriction digest was treated with 8-μL RNAsecure (Ambion) and incubate at 60° C. for ten minutes. RNAsecure treated DNA was purified using the MINIELUTE PCR cleanup kit from Qiagen and eluted in 10-μL of RNAase free injection buffer (5 mM Tris Cl, pH 7.5; 0.1 mM EDTA). Synthetic mRNA were produced using the mMESSAGE MACHINE T3 kit (Ambion) using 1 ug of linearized template and 1.5 hours incubate at 37° C. After 15 minutes treatment with Turbo DNAase the mRNA was purified using the Ambion MEGACLEAR kit and eluted 2× with 50-μL of heated H2O.

### C. Microinjection of TALENs Pairs

RNA encoding each TALEN arm were combined and resuspended in nuclease free water at a concentration of 10-200 ng/μL. 5-20-μL were injected into one cell stage tilapia embryos. Injected embryos survival was measured at 6 days post fertilization against a non-injected control group. RNA concentration giving a 50% rate of survival was used for repeat/standard injections to generate Knock outs. To confirm that injected embryos died from TALENs induced mutagenesis, deformed embryos were collected and mutation at the target site was investigated using a QPCR melt profile analysis.

### D. Tissue Collection and DNA Extraction of Control and RNA Treated Tilapia.

Six day old RNA treated embryos (deformed) were dechorionated anesthetized and the yolk sac was removed using a razor blade. Embryonic tissue was digestion overnight in lysis buffer; 10 mM Tris, 10 mM EDTA, 200 mM NaCl, 0.5% SDS, 100 mg/ml proteinase K and extracted with automated Research X-tractor, Corbett robotic system using whatman™ unifilter 800, 96 well plates (GE Healthcare, UK). Embryos that survived microinjection and developed normally (from groups with ˜50% survival rate) were raised to 1 month of age, anaesthetized; fin clipped and place in individual jars while their fin DNA was analyzed (overnight digestion in lysis buffer followed by DNA extraction as described above). Sperm was stripped from G0 males carrying somatic mutations at the kiss or kissR loci and gDNA extracted using DNAzol Reagent (Life Technologies) following standard procedure. Extracted DNA was resuspended in 30 μl of MQ H2O.

### E. Identification of Mutation by QPCR

Real-time qPCR was performed ROTOR-GENE RG-3000 REAL TIME PCR SYSTEM (Corbett Research). 6-μL genomic DNA (gDNA) template (diluted at 1 ng/ml) was used in a total volume of 15 μL containing 0.4 μM concentrations each of the forward and reverse primers and 7.5 μL of 2× Brilliant II SYBR GREEN QPCR MASTER MIX (Agilent Technologies). qPCR primers were designed using DNAstar software (Table 1). The qPCR was performed using 40 cycles of 15 seconds at 95° C., 60 seconds at 60° C., followed by melting curve analysis to confirm the specificity of the assay (67° C. to 97° C.). In this approach, to detect the occurrence of a DNA polymorphism at the targeted kiss and kissR loci, short PCR amplicons (approx 100-140 bp) that include the region of interest are generated from a gDNA sample, subjected to temperature-dependent dissociation (melting curve). When TALEN-induced polymorphisms are present in the template gDNA, heteroduplex as well as different homoduplex molecules will be formed. The presence of multiple forms of duplex molecules is detected by Melt profile, showing whether duplex melting acts as a single species or more than one species. Generally, the symmetry of the melting curve and melting temperature infers on the homogeneity of the dsDNA sequence and its length. For example, if small insertion or deletions resulting from repair of TALENs-induced DSBs by NHEJ are generated then that melting temperature will positively correlate to the length of the deletion or insertion, proportionally to the energy required to break the base-base hydrogen bonding. If multiple forms of duplex molecules are present, the temperature dependant denaturation will detect together the most instable heteroduplex and the most stable homoduplex giving a modified (dissymmetric) melt profile. The Melt analysis is performed by comparison with reference DNA sample (from non-injected tilapia control or plasmid containing the genomic region of interest) amplified in parallel with the same master mix reaction. In short, variation in melt profile distinguishes sequences carrying TALEN induced mutation from wild type sequence, thus facilitating the screen.

### F. Calculating Mutation Rates in Somatic Cells or Germ Cells of Microinjected Tilapia and Characterization of TALEN Induced Mutations.

Fish whose somatic or germ cells gDNA produced asymmetric qPCR melt profiles (candidate mutant) were further analyzed to measure the mutagenic frequency. Genomic PCR products containing the target site (442 bp for Kiss and 720 bp for KissR) were obtained from fin-DNA or sperm-DNA. The PCRs were carried out in a 25-μL reaction mixture, which contained 120-180 ng template gDNA, 0.1 μl of Platinum Taq DNA polymerase, 0.2 mM dNTPs, 1× Taq DNA polymerase buffer, 2 mM Mg2+, and 0.2 μM of each primer. DNA amplification was done under the following conditions: 95° C. for 5 min, followed by 35 cycles of 94° C. for 30 s, 55° C. for 30 s, and 72° C. for 45 s, with a final extension at 72° C. for 2 min. The PCR products were cloned into TOPO 2.1 TA vector (Invitrogen), and transformed into competent E. coli cells (ONE SHOT, Top 10F′, Invitrogen). Transformant colonies were randomly picked with a sterile pipette tips and transferred directly onto individual qPCR reaction tubes before replating on selective agar media. qPCR were performed using primers that span the TALENs target sites of interest (100-140 bp amplicons). QPCR reactions showing specific product amplification were compared against a reference DNA sample control (wild type sequence) to identify melt profile variants (FIG. 10 panels c and d). DNA mutation rate was calculated as the number of mutant sequences (colonies with variant melt) divided by the total number of sequences analyzed multiplied by 100. To visualize the mutations present at the target loci, clones representing individual somatic or sperm cells were displayed in a Scatter plot of Ct versus Melt temperatures (see FIG. 10 panel d for example). In these graphs, each E. Coli colony is represented by a data point (x, y), with x representing its Ct and y representing its melt. Individual colonies carrying identical sequences should display similar melting temperature. Colonies showing variant melting temperature were grown overnight and their plasmid extracted and purified (MINIPREPARATION kit, QUIAGEN). The region containing the TALENs target site were then sequenced using selected primers for the kiss and kissR regions, as indicated. To characterize mutations in F1 and F2 fish, the 442 bp and 702 bp amplicons containing the target kiss1.1a and KissRE3 loci were purified on silica-membrane-based spin column (QIAQUICK PCR PURIFICATION KIT, QIAGEN). The purified PCR were directly sequenced using an internal primer (KissRF).

### G. Founder Screen

Gametes were stripped from all putative founders and F1 embryos were produced from in vitro fertilization with gametes collected from WT stock. 3 weeks post-fertilization, F1 progeny were fin-clipped and held separately in individual jar. Fin DNA was extracted as previously describe (see Tissue collection and DNA extraction section above) and adjusted to 1 ng/μl using a spectrophotometer NANODROP ND1000). In general, 10-20 juveniles from each potential founder were screened by QPCR using the melt analysis strategy described above. For sequence confirmation, genomic DNAs from single embryo/juvenile were amplified and the PCR product submitted to sequencing after purification.

Sequencing chromatography of PCR showing two simultaneous reads are indicative of the presence of indels. The start of the deletion or insertion typically begins when the sequence read become divergent. The dual sequences are than carefully analyze to detect unique nucleotide reads (see FIG. 12 panel a). The pattern of unique nucleotide read is then analyzed against series of artificial single read patterns generated from shifting the wild type sequence over itself incrementally.

### H Mutagenic Potency of Engineered TALENs

Engineered TALENs and synthetic capped mRNA encoding each heterodimeric TALENs together was injected at various concentrations from 10 to 250 ng/μl into 1-cell stage tilapia embryos. We then observed the injected embryos at 6 days post fertilization (dpf). Embryos injected with less than 10 ng of TALENs developed normally while a dose of 200 ng (Kiss1a) and 100 ng (KissRE3) generated up to 50% of dead or deformed embryos. Dose of 250 ng for kiss1.1b and kissRE2 generated less the 30% mortality. On day five, injected embryos were separated between those that developed normally from those with morphological deformities. To check for evidence of mutations, genomic DNA was isolated from a pool of 3 deformed embryos for each TALENs treated group and from 3 normal embryos from a non-injected control group. Genomic DNA was used for QPCR melt analysis of the target loci. Asymmetric melt profile were found in the pool of embryos treated with TALENs targeting the kiss1.1a and kissRE3 loci (data not shown) but not in embryos treated with the other 2 TALENs pairs.

To confirm the presence of mutation, 20-40 normally developed juveniles in each group were assayed by QPCR melt analysis. None of the fish injected with TALEN KissR-E2 and Kiss1.1b mRNA produced variant melt suggesting that either no mutation were created or that the mutation did not produce detectable melt variation. Nevertheless, a total of 8 fish producing variant melt profiles were found, 4 for each kiss1.1a and KissRE3 loci (FIG. 10 panel a, and panel b). To confirm that the observed melt variation results from a mixture of wild-type and NHEJ products with micro-insertion or deletion at the target site, each target region (442 bp for Kiss and 702 bp for KissR) was amplified in a PCR reaction. The resulting PCR fragments were cloned into Topo TA vectors and transformant colonies screened by direct real time-PCR. For each fish tested, between 14 and 21 E. Coli transformant colonies were hand-picked (randomly) and added directly (without DNA purification) to the Q-PCR reaction mixture.

Colonies carrying mutated alleles were identified by comparison to the wild-type unmodified sequence. High frequencies of colonies with variant melt profiles ranging from 50-91% were detected (FIG. 10 panel's c and d).

To characterize some of these lesions, the plasmid from clones that produced variant amplicons was extracted and the PCR insert was sequenced. Between 4 and 7 clones were sequenced for each TALENs treated group and all but one carried mutated alleles. A total of fourteen different somatic mutations in the kiss and kissr genes were detected from all 8 TALENs treated fish (eight at the Kiss1.1 a loci and six at KissRE3 loci). Nine different nucleotide deletions, two insertions, and three combinations of nucleotide insertions and deletions were observed (FIG. 11 panels a, and b). A deletion/insertion of as little as 3 bp was detectable by RT-PCR melt analysis. It was observed that TALENs induced mutation occurs multiple times in an RNA treated fish resulting in mosaic somatic mutations (see table below).

It was found that more than 95% of the sequences from colonies showing melt variation carry a mutation indicating that DNA mutation rate can be approximated by measuring the frequency of clones producing variant melt. Thus, the rate of mutation was calculated to be between 35% and 91% depending on the fish. This result indicates the highly efficient introduction of targeted indels at the expected genomic locations.

The table, Summary of the results of somatic mutation screen, shows results for TALENs-injected tilapia. The second column describes the mutant sequences identified in somatic cells, including the sizes of the indels (+, insertion; −, deletion) and the resulting protein sequence modification are shown inside the parentheses. In the last column, the estimated rate of somatic mutation for each fish was calculated from the frequency of colonies producing variant melting temperature.

TABLE
Summary of the results of somatic mutation screen

| | | % of mosaic somatic |
|---|---|---|
| | | mutations (n = total |
| Fish | | number of colonies |
| reference | Mutation type | screened) |
| Kiss17 | +10 nt(frame shift/stop); +4 nt | 73% (n = 22) |
| | (frame shift/stop); Δ12 nt (−4AA) | |
| | and Δ18 nt (−6AA) | |
| Kiss19 | Δ12 nt (−4AA) | 48% (n = 21) |
| Kiss20 | Δ16 nt (frame shift/stop); Δ12 nt | 91% (n = 23) |
| | (−4AA) | |
| Kiss41 | Δ4 nt (frame shift/stop); Δ12 nt | 85% (n = 14) |
| | (−4AA and F > C); Δ12 nt (−4AA) | |
| RE3-1 | Δ10 nt (frame shift/stop); Δ7 nt | 35% (n = 21) |
| | (frame shift/stop) | |
| RE3-4 | Δ3 nt (frame shift/stop); Δ26 nt | 85% (n = 14) |
| | (frame shift/stop) | |
| RE3-6 | Δ5 nt (frame shift/stop); Δ14 nt | 63% (n = 16) |
| | (frame shift/stop) | |
| RE3-11 | Δ7 nt (frame shift/stop) | 66% (n = 21) |

### I. Sequence Analysis of TALENs Mutations

Of the different types of nucleotide mutation, five and six caused a frameshift leading to the generation of premature stop codons in the kiss and kissr gene respectively. Also, there was a high frequency of 12 nt deletions at the Kiss1.1a loci which occurred independently in all 4 TALENs treated fish. This mutation result in the loss of 4 amino acids (AA).

F0 TALENs-mutated tilapia were raised to sexual maturity and their sexes were determined. To show that TALENs treated fish can induce heritable mutations; genomic DNA was extracted from the semen's of each spermiating animals and screened. The frequency of sperm carrying mutation was determined by the frequency of clones showing variant melt profiles as previously described. To characterize the sperm associated lesions, the plasmids from colonies with variant melt was extracted and sequenced. Germline mutation frequency ranging from 50% to 91% was observed. Sequences revealed the existence of multiple indels in each fish germline.

TABLE
Sequencing

| | | % of mosaic somatic |
|---|---|---|
| | | mutations (n = total |
| Male Fish | | number of colonies |
| reference | Mutation type | screened) |
| Kiss17 | Δ12 nt (−4AA) and Δ18 nt (−6AA) | 50% (n = 20) |
| Kiss19 | Δ12 nt (−4AA); +3 nt (+1AA) | 65% (n = 30) |
| Kiss20 | Δ16 nt (frame shift/stop); Δ12 nt | 91% (n = 23) |
| | (−4AA) | |
| RE3-4 | Not sequenced | 88% (n = 18) |
| RE3-6 | Not sequenced | |

J. Analysis of Germ Line Mutations at the kiss and kissR Loci.

To further demonstrate that Kiss and kissR TALENs effectively induced mutation in the germ line, the 8 founders were intercrossed with wild-type stocks. All 8 TALENs treated fish were fertile and produced viable clutches of embryos. These progeny were raised and screened for the presence of mutated alleles. All 8 founders could transmit heritable mutations. The analysis first showed that the fraction of progeny carrying putative mutation ranged between 16% and 90% as gauged by QPCR melt profile analysis of F1 fin-DNA extracts. As expected, there was a positive correlation between the extent of mosaicism in the TALENs treated parent and the frequency of progeny carrying a mutation. Analysis of selected gene sequences producing deformed melt profile all revealed a range of induced indel mutations, some of which were previously found in somatic tissue of the founders (FIG. 12 panel b). Furthermore, sequencing of F1 fish producing wild type melt all revealed wild type sequences. More than one type of heritable mutation from a single founder was often observed, suggesting that those mutations occurred independently in different germ cells within the same animal. Inherited mutations included deletions ranging in size from 3 to 18 bp (FIG. 12 panel b). In the progeny of all 4 kiss mutant founders, the only inherited mutations were deletions of 12 nt and 18 nt which resulted in the loss of four and six AA. Although, those deletions did not result in frameshift mutations they remove either one or three AA at the most C-terminal region of the kiss-10 peptide (FIG. 12 panel c). Because this core sequence was found essential and sufficient for the activation of the kissR signaling pathway throughout vertebrates, those mutations would produce a loss of function phenotype. Also identified was a frame shift mutation at the kissRE3 loci which was not previously isolated in the founder. All frameshift mutations resulted in a premature stop codon removing between 172 AA and up to 215 AA (Δ7 nt, FIG. 12 panel c) from the C-terminal portion of the KissR protein. These mutations, which remove as much as 57% of the protein sequence, will inactivate the gene function. All kiss and kissr mutations identified among the juveniles F1 offspring were viable in the heterozygous state.

TABLE
Summary of founder screening results. In the last column of each table, the numbers
of embryos carrying indel mutations are shown outside of the parentheses, and the
sizes of the indels are shown inside the parentheses. +, insertion; −, deletion.

| Fish | % F1 with putative | #F1 sequenced | | |
|---|---|---|---|---|
| reference | mutations (n = total | (Variant + WT | # of mutants | |
| (sex) | number of F1 screened) | melt) | identified | Mutation type |
| Kiss17 (♂) | 66% (n = 30) | 13 + 2 | 13 | 7 {Δ12 nt (−4AA)} and 6 {Δ18 nt |
| | | | | (−6AA)} |
| Kiss19 (♂) | 49% (n = 37) | 10 + 2 | 10 | 10 {Δ12 nt (−4AA)} |
| Kiss20 (♂) | 73% (n = 29) | 12 + 2 | 12 | 12{Δ12 nt (−4AA)} |
| Kiss41 (♀) | 16% (n = 38) | 6 + 2 | 6 | 6{Δ12 nt (−4AA)} |
| | % F1 carrying putative | #F1 sequenced | | |
| Fish | mutations (n = total | (Variant + WT | #of mutants | |
| reference | number of F1 screened) | melt) | identified | Mutation type |
| RE3-1 (♀) | 29% (n = 44) | 19 + 2 | 19 | 10{Δ3 nt (−1AA, R > Q); 8{Δ11 nt |
| | | | | (frame shift/stop)}; 1 {Δ8 nt, |
| | | | | (frameshift/stop) } |
| RE3-4 (♂) | 90% (n = 22) | 10 + 2 | 10 | 9{Δ9 nt (−3AA)}; 1{Δ5 nt, (frame |
| | | | | shift/stop)} |
| RE3-6 (♂) | | | | |
| RE3-11 (♀) | 63% (n = 35) | 11 + 2 | 11 | 10{Δ7 nt (frame shift/stop); |
| | | | | 1{Δ5 nt (frame shift/stop)} |

### K. F1 and F2 Generations

F1 heterozygous mutants showed no morphological defect as they continued to develop, and all differentiated into fertile adult of both sex. The absence of a reproductive phenotype in sexually mature F1 generation is not unexpected given the presence of a wild type allele of each targeted gene in all somatic cells of selected mutant. The characterization of an inactivation phenotype is only possible in the F2 generation in fish carrying the associated loss-of-function mutation in the homozygous (or compound heterozygous) state. To generate homozygous mutation, sperm and eggs collected from F1 heterozygous mutant were used to produce F2 generations, which are being grown; these F2 generations are, at the time of filing, at an age prior to the time of normally expected sexual maturity.

### Further Description

1. A genetically modified livestock animal comprising a genome that comprises inactivation of a neuroendocrine gene selective for sexual maturation, with the inactivation of the gene preventing the animal from becoming sexually mature. 2. The livestock animal of 1 wherein the inactivation of the gene comprises an insertion, deletion, or substitution of one or more bases in a sequence encoding the sexual maturation gene and/or a cis-regulatory element thereof. 3. The livestock animal of 1 wherein the inactivated gene is inactivated by: removal of at least a portion of the gene from a genome of the animal, alteration of the gene to prevent expression of a functional factor encoded by the gene, or a trans-acting factor. 4. The livestock animal of 3 wherein the gene is inactivated by the trans-acting factor, said trans-acting factor being chosen from the group consisting of interfering RNA and a dominant negative factor, with said trans-acting factor being expressed by an exogenous gene or an endogenous gene. 5. The livestock animal of 4 wherein the trans-acting factor comprises a dominant negative for GPR54. 6. The livestock animal of 1-5 wherein the inactivation of the gene is under control of an inducible system. 7. The livestock animal of 6 wherein the inducible system comprises a member of the group consisting of Tet-On, Tet-Off, Cre-lox, and Hif1 alpha. 8. The livestock animal of 1-7 wherein the animal is chosen from the group consisting of cattle, swine, sheep, chicken, goats, and fish. 9. The livestock animal of 1-8 wherein the sexual maturation gene is chosen from the group consisting of Gpr54, Kiss1, and GnRH11. 10. The livestock animal of 1-9 wherein the animal further expresses a trait as a result of expression of a recombinant protein. 10a. The livestock animal of 1-10 wherein the animal further expresses an exogenous recombinant protein. 11. The livestock animal of 10 wherein the trait is chosen from the group consisting of production traits, type traits, and workability traits. 12a. The livestock animal of 1-11 being sexually immature at an age that a wild type animal of the same species is sexually mature. 12b. The livestock animal of 1-11 being genetically unable to mature without a treatment. 13. A genetically modified livestock animal comprising a genome that is heterozygous for an inactivation of a neuroendocrine gene selective for sexual maturation, wherein progeny homozygous for the inactivated gene are thereby prevented from becoming sexually mature. 14. The animal of 13 wherein the sexual maturation gene is chosen from the group consisting of Gpr54, Kiss1, and GnRH11. 15. An in vitro organism chosen from the group consisting of a cell or an embryo, the in vitro organism comprising a genome that comprises an inactivation of a sexual maturation gene. 16. The organism of 15 being a cell or embryo chosen from the group consisting of cattle, swine, sheep, chicken, goats, rabbit, and fish. 17. The organism of 15-16 wherein the inactivation is in a gene chosen from the group consisting of Gpr54, KiSS1, and GnRH11. 18. A process of making a livestock animal comprising introducing, into an organism chosen from the group consisting of a livestock cell and a livestock embryo, an agent that specifically binds to a chromosomal target site of the cell and causes a double-stranded DNA break to inactivate a neuro endocrine gene selective for sexual maturation, with the agent being chosen from the group consisting of a TALEN, a zinc finger nuclease, Cas9/CRISPR and a recombinase fusion protein. 19. The process of 18 wherein the agent is a TALEN or a TALEN pair that comprises a sequence to specifically bind the chromosomal target site, and creates the double stranded break in the gene or creates the double stranded break in the chromosome in combination with a further TALEN that creates a second double stranded break with at least a portion of the gene being disposed between the first break and the second break. 20. The process of 18-19 further comprising co-introducing a recombinase into the organism with the TALEN or TALENs. 21. The process of 19 wherein a transgene expressing the agent is placed in a genome of the organism. 22. The process of 18-21 wherein the introducing the agent into an organism comprises a method chosen from the group consisting of direct injection of the agent as peptides, injection of mRNA encoding the agent, exposing the organism to a vector encoding the agent, and introducing a plasmid encoding the agent into the organism. 23. The process of 18-22 wherein the agent is the recombinase fusion protein, with the process comprising introducing a targeting nucleic acid sequence with the fusion protein, with the targeting nucleic acid sequence forming a filament with the recombinase for specific binding to the chromosomal site. 24. The process of 18-23 wherein the recombinase fusion protein comprises a recombinase and Ga14. 25. The process of 18-24 further comprising introducing a nucleic acid into the organism, wherein the nucleic acid is inserted into the genome of the organism at a site of the double-stranded break or between the first break and second break. 25a. The process of 18-24 further comprising introducing an exogenous nucleic acid template having a sequence into the organism, with the genome of the organism at a site of the double-stranded break receiving the sequence. The exogenous template can be copied or actually inserted into the genome, with the result being the same, regardless of the theories about it being one or the other mechanism. The result is that the genome has the sequence of the template. 26. The process of 18-25 wherein the nucleic acid comprises a member of the group consisting of a stop codon, a reporter gene, and a reporter gene cassette. 27. The process of 18-26 further comprising cloning the animal from the organism. 28. The process of 18-27 wherein the animal is chosen from the group consisting of cattle, swine, sheep, chicken, goats, rabbit, and fish. 29. The process of 18-28 wherein the sexual maturation gene is chosen from the group consisting of Gpr54, Kiss1, and GnRH11. 30. The livestock animal of 18-29 wherein the inactivation of the gene is under control of an inducible system. 31. A process of raising a livestock animal comprising administering an agent to an animal for sexual maturation of the animal, with the agent compensating for a genetic inability of the animal to sexually mature. 32. The process of 31 wherein the agent comprises a gonadotropin or a gonadotropin analogue. 33. The process of 31-32 further comprising breeding the sexually mature animal to produce progeny. 34. The process of claim 31-33 wherein the genetic inability of the animal to mature is a result of a genetically inactivated neuroendocrine gene selective for sexual maturation. 35. The process of 34 wherein the inactivated gene is chosen from the group consisting of Gpr54, Kiss1, and GnRH11. 36. The process of 34 wherein the inactivated gene is inactivated by: removal of at least a portion of the gene from a genome of the animal, alteration of the gene to prevent expression of a functional factor encoded by the gene, or a trans-acting factor. 37. The process of 31-36 wherein the animal is chosen from the group consisting of cattle, swine, chicken, sheep, fish, rabbit, and goats, the administration of the agent to the animals takes place in a treatment facility, and the progeny are distributed from the treatment facility to a plurality of locations to be raised.

TABLE 1
Frequencies for recovery of colonies with HDR alleles

| | | | Mutation | | aa | Day 3% | | Bi-allelic |
|---|---|---|---|---|---|---|---|---|
| Reagent | ID | Species | type | nt change | change | HDR | HDR+ (%) | HDR+ (%) |
| TALEN | ssLDLR2.1^{a} | Pig ♀ | Ins/FS | 141(ins4) | 47ΔPTC | 38 | 55/184 (30) | 4/184 (2) |
| TALEN | ssDAZL3.1^{b} | Pig ♂ | Ins/FS | 173(ins4) | 57ΔPTC | 25 | 34/92 (37) | 8/92 (9) |
| TALEN | ssDAZL3.1^{Rep} | Pig ♂ | Ins/FS | 173(ins4) | 57ΔPTC | 30 | 42/124 (34) | 7/124 (6) |
| TALEN | ssAPC14.2^{b} | Pig ♂ | Ins/FS | 2703(ins4) | 902ΔPTC | 48 | 22/40 (55) | 4/40 (10) |
| TALEN | ssAPC14.2^{Rep} | Pig ♂ | Ins/FS | 2703(ins4) | 902ΔPTC | 50 | 57/96 (60) | 19/96 (20) |
| TALEN | ssAPC14.2^{Ld} | Pig ♂ | Ins/FS | 2703(ins4) | 902ΔPTC | 34 | 21/81 (26) | 1/81 (1) |
| TALEN | ssTp53 | Pig ♂ | Ins/FS | 463(ins4) | 154ΔPTC | 22 | 42/71 (59) | 12/71 (17) |
| TALEN | ssRAG2.1 | Pig ♂ | Ins/FS | 228(ins4) | 76ΔPTC | 47 | 32/77 (42) | 13/77 (17) |
| TALEN | btRosa1.2^{c} | Cow ♂ | Ins/mloxP | ins34 | NA | 45 | 14/22 (64) | 7/22 (32) |
| TALEN | ssSRY3.2 | Pig ♂ | Ins/mloxP | ins34 | NA | 30 | ND | ND |
| TALEN | ssKissR3.2 | Pig ♂ | Ins/FS | 322(ins6) | 107ΔPTC | 53 | 57/96 (59) | 17/96 (18) |
| | | | | 323(del2) | | | | |
| TALEN | btGDF83.1 | Cow ♂ | del/FS | 821(del11) | FS | ~10 | 7/72 (10) | 2/72 (3) |
| TALEN | ssEIF4GI14.1 | Pig ♂ | SNPs | G2014A | N672D | 52 | 68/102 (67) | 40/102 (39) |
| | | | | T2017C | L673F | | | |
| | | | | C2019T | | | | |
| TALEN | btGDF83.6N | Cow ♂ | SNPs | G938A | C313Y | 18 | 8/94 (9) | 3/94 (3) |
| | | | | T945C | | | | |
| TALEN | btGDF83.6N^{d} | Cow ♂ | SNP | G938A | C313Y | NA | 7/105 (7) | 2/105 (2) |
| TALEN | ssP65.8 | Pig ♂ | SNP | T1591C | S531P | 18 | 6/40 (15) | 3/40 (8) |
| TALEN | ssP65.8^{Rep} | Pig ♂ | SNP | T1591C | S531P | 7 | 9/63 (14) | 5/63 (8) |
| TALEN | ssGDF83.6^{d} | Pig ♂ | SNP | G938A | C313Y | NA | 3/90 (3) | 1/90 (1) |
| TALEN | caFecB6.1 | Goat ♂ | SNP | A747G | Q249R | 17 | 17/72 (24) | 3/72 (4) |
| TALEN | caCLPG1.1 | Goat ♂ | SNP | A→G | Non- | 4 | ND | ND |
| | | | | | coding | | | |
| CRISPR | ssP65 G1s | Pig ♂ | SNP | T1591C | S531P | 6 | 6/96 (6) | 2/96 (2) |
| CRISPR | ssP65 G2a | Pig ♂ | SNP | T1591C | S531P | 5 | 2/45 (4) | 0/45 |
| CRISPR | APC14.2 G1a | Pig ♂ | Ins/FS | 2703(ins4) | 902ΔPTC | 32 | ND | ND |

1. A genetically modified livestock animal comprising a genome that comprises inactivation of a neuroendocrine gene selective for sexual maturation, with the inactivation of the gene preventing the animal from becoming sexually mature.

2. The livestock animal of claim 1 wherein the inactivation of the gene comprises
an insertion, deletion, or substitution of one or more bases in
a sequence encoding the sexual maturation gene and/or a cis-regulatory element thereof.

3. The livestock animal of claim 1 wherein the gene is inactivated by the trans-acting factor, said trans-acting factor being chosen from the group consisting of interfering RNA and a dominant negative factor, with said trans-acting factor being expressed by an exogenous gene or an endogenous gene.

4. The livestock animal of claim 1 wherein the inactivation of the gene is under control of an inducible system.

5. The livestock animal of claim 1 wherein the animal is chosen from the group consisting of cattle, swine, sheep, chicken, goats, and fish.

6. The livestock animal of claim 1 wherein the sexual maturation gene is chosen from the group consisting of Gpr54, Kiss1, and GnRH11.

7. The livestock animal of claim 1 wherein the animal further expresses an exogenous recombinant protein.

8. The livestock animal of claim 1 being genetically unable to mature without a treatment.

9. A genetically modified livestock animal comprising a genome that is heterozygous for an inactivation of a neuroendocrine gene selective for sexual maturation, wherein progeny homozygous for the inactivated gene are thereby prevented from becoming sexually mature.

10. The animal of claim 9 wherein the neuroendocrine gene is chosen from the group consisting of Gpr54, Kiss1, and GnRH11.

11. An in vitro organism chosen from the group consisting of a cell or an embryo, the in vitro organism comprising a genome that comprises an inactivation of a sexual maturation gene.

12. The organism of claim 11 being a cell or embryo chosen from the group consisting of cattle, swine, sheep, chicken, goats, rabbit, and fish.

13. The organism of claim 11 wherein the inactivation is in a gene chosen from the group consisting of Gpr54, KiSS1, and GnRH11.

14. A process of making a livestock animal comprising
introducing, into an organism chosen from the group consisting of a livestock cell and a livestock embryo, an agent that specifically binds to a chromosomal target site of the cell and causes a double-stranded DNA break to inactivate a neuroendocrine gene selective for sexual maturation, with the agent being chosen from the group consisting of a TALEN, a zinc finger nuclease, Cas9/CRISPR and a recombinase fusion protein.

15. The process of claim 14 comprising co-introducing a recombinase into the organism with a TALEN.

16. The process of claim 14 wherein a transgene expressing the agent is placed in a genome of the organism.

17. The process of claim 16 wherein the agent is the recombinase fusion protein, with the process comprising introducing a targeting nucleic acid sequence with the fusion protein, with the targeting nucleic acid sequence forming a filament with the recombinase for specific binding to the chromosomal site.

18. The process of claim 14 further comprising introducing a nucleic acid template having a sequence into the organism, with the genome of the organism at a site of the double-stranded break receiving the sequence.

19. The process of claim 14 further comprising cloning the animal from the organism.

20. The process of claim 14 wherein the animal is chosen from the group consisting of cattle, swine, sheep, chicken, goats, rabbit, and fish.

21. The process of claim 14 wherein the sexual maturation gene is chosen from the group consisting of Gpr54, Kiss1, and GnRH11.

22. The process of claim 14 wherein the inactivation of the gene is under control of an inducible system.

23. A process of raising a livestock animal comprising
administering an agent to an animal for sexual maturation of the animal, with the agent compensating for a genetic inability of the animal to sexually mature.

24. The process of claim 23 wherein the agent comprises a gonadotropin or a gonadotropin analogue.

25. The process of claim 23 wherein the genetic inability of the animal to mature is a result of a genetically inactivated neuroendocrine gene selective for sexual maturation.

26. The process of claim 25 wherein the inactivated gene is chosen from the group consisting of Gpr54, Kiss1, and GnRH11.


SEQUENCE LISTING (86 sequences)

SEQ ID NO: 1 - DNA, 25 bp, Artificial Sequence
  Primer
ttcaccagaagacaaggagaattgc

SEQ ID NO: 2 - DNA, 27 bp, Artificial Sequence
  Primer
taaggccaattactgctctggagacta

SEQ ID NO: 3 - DNA, 25 bp, Artificial Sequence
  Primer
ccttgaggtaggagagtgttttggg

SEQ ID NO: 4 - DNA, 28 bp, Artificial Sequence
  Primer
cctttttagaagtcaaggtaacagacac

SEQ ID NO: 5 - DNA, 25 bp, Artificial Sequence
  Primer
ttgattggagacatctttgtgggag

SEQ ID NO: 6 - DNA, 27 bp, Artificial Sequence
  Primer
taaggccaattactgctctggagatta

SEQ ID NO: 7 - DNA, 24 bp, Artificial Sequence
  Primer
gcatcgagattctgtcacaatcaa

SEQ ID NO: 8 - DNA, 23 bp, Artificial Sequence
  Primer
aaggatgtcagcacctctctggg

SEQ ID NO: 9 - DNA, 25 bp, Artificial Sequence
  Primer
acccacccggactctactcctacca

SEQ ID NO: 10 - DNA, 50 bp, Artificial Sequence
  Expression Vector
acaaccctctcagccttcgctttgggaaacgctacaatggctacatttac

SEQ ID NO: 11 - DNA, 54 bp, Artificial Sequence
  Expression Vector
cgctttgggaaacgctacaatggctacatttacagaagagctgttaaaagagcc

SEQ ID NO: 12 - DNA, 57 bp, Artificial Sequence
  Expression Vector
ccccttcaccgccaccctttaccccctccctggatggatctttggcaacttcatgtg

SEQ ID NO: 13 - DNA, 55 bp, Artificial Sequence
  Expression Vector
ctaccccctgaaatctcttcggcaccgaacccccaaagtagccatgattgtcagc

SEQ ID NO: 14 - DNA, 21 bp, Artificial Sequence
  Primer
gccactgacatcatcttcttg

SEQ ID NO: 15 - DNA, 20 bp, Artificial Sequence
  Primer
gaaacagaaagttgaagtgg

SEQ ID NO: 16 - DNA, 23 bp, Artificial Sequence
  Primer
tcaccctgactgctatgagtgga

SEQ ID NO: 17 - DNA, 23 bp, Artificial Sequence
  Primer
atgagtcagtcgataatgacacg

SEQ ID NO: 18 - DNA, 21 bp, Artificial Sequence
  Primer
ttatgcaaaagaagaaaggtg

SEQ ID NO: 19 - DNA, 24 bp, Artificial Sequence
  Primer
gcagagttcgacctactttcattg

SEQ ID NO: 20 - DNA, 24 bp, Artificial Sequence
  Primer
tatacatagcccccattttcagtg

SEQ ID NO: 21 - DNA, 19 bp, Artificial Sequence
  Primer
ggcagcaggtaggcagcaa

SEQ ID NO: 22 - DNA, 23 bp, Artificial Sequence
  Primer
gtcctctgcattcaggagaacag

SEQ ID NO: 23 - DNA, 24 bp, Artificial Sequence
  Primer
ctaaaagtattttatttacatagt

SEQ ID NO: 24 - DNA, 21 bp, Artificial Sequence
  Primer
aggcagctcctttgcaatgat

SEQ ID NO: 25 - DNA, 21 bp, Artificial Sequence
  Primer
agagaagggtgaaaacttttt

SEQ ID NO: 26 - DNA, 59 bp, Bos primigenius
tgtgatgaacactccacagaatctcgatgctgtcgttaccctctaactgtggattttga

SEQ ID NO: 27 - DNA, 48 bp, Artificial Sequence
  Modified Allele
tgtgacagaatctcgatgctgtcgttaccctctaactgtggattttga

SEQ ID NO: 28 - DNA, 59 bp, Artificial Sequence
  Modified Allele
ttgggcttgattgtgacagaatctcgatgctgtcgttaccctctaactgtggattttga

SEQ ID NO: 29 - DNA, 61 bp, Sus scrofa
tagatggatgaaaccgaaattagaagtttctttgctagatatggttcagtaaaagaagtga

SEQ ID NO: 30 - DNA, 65 bp, Artificial Sequence
  Modified Allele
tagacggatgaaaccgaaattagaagttggatcctttgctagatatggttcagtaaaaggagtga

SEQ ID NO: 31 - DNA, 65 bp, Artificial Sequence; identical to SEQ ID NO: 30
  Modified Allele

SEQ ID NO: 32 - DNA, 29 bp, Artificial Sequence
  Modified Allele
tagatggatgaaaccgaaattagaagtga

SEQ ID NO: 33 - DNA, 39 bp, Artificial Sequence
  Modified Allele
tagatggatgaaaccgatatggttcagtaaaagaagtga

SEQ ID NO: 34 - DNA, 35 bp, Artificial Sequence
  Modified Allele
tagatggactagatatggttcagtaaaagaagtga

SEQ ID NO: 35 - DNA, 61 bp, Sus scrofa
tcatggaagaagtatcagccattcatccctcccaggaagacagaaattctgggtcaaccac

SEQ ID NO: 36 - DNA, 65 bp, Artificial Sequence
  Modified Allele
tcacggaagaagtatcagccattcatccctcccagtgaagcttacagaaattctgggtcagccac

SEQ ID NO: 37 - DNA, 65 bp, Artificial Sequence
  Modified Allele
tcatggaagaagtatcagccattcatccctcccagtgaagcttacagaaattctgggtcaaccac

SEQ ID NO: 38 - DNA, 64 bp, Artificial Sequence
  Modified Allele
tcatggaagaagtatcagccattcatccctccccccaggaagacagaaattctgggtcaaccac

SEQ ID NO: 39 - DNA, 65 bp, Artificial Sequence
  Modified Allele
tcatggaagaagtatcagccattcatccctcccagtgaagcttacagaaattctgggtcagccac

SEQ ID NO: 40 - DNA, 61 bp, Sus scrofa; identical to SEQ ID NO: 35

SEQ ID NO: 41 - DNA, 65 bp, Artificial Sequence
  Modified Allele
tcacggaagaagtatcagccattcatccctcccagtgaagcttacagaaattctgggtcaaccac

SEQ ID NO: 42 - DNA, 58 bp, Artificial Sequence
  Modified Allele
tcatggaagaagtatcagccattcatccctccgaagacagaaactctgggtcaaccac

SEQ ID NO: 43 - DNA, 90 bp, Sus scrofa
gtgctgcgtgccctttactgctctactctaccccctaccagcctgggtgctgggcgacttcatgtgcaagttcctcaactacatccagca

SEQ ID NO: 44 - DNA, 90 bp, Artificial Sequence
  Modified Allele
gtgctgcgtgccctttactgctctactctaccccctaccagcctnngnnngtgctgggcgacttcatgtgcaagttcctcaactacatcc

SEQ ID NO: 45 - DNA, 630 bp, Oreochromis Niloticus
acatggggagacaaacaatttttggatctggtgctgaaagatgagactactggctttggctgtggtttgcgctctcattgctatccaggatggagggagtgtgggagcagctctgccaggagtcgaccctgcacagagaacacatgcaacaggagcagtgtcctctgcattcaggagaacagcaggcgacttcctggcagaggatcccagcctctgcttttccctgagagagaacgaggaccagaggcagctcctttgcaatgatcgcagaagtaatttcaactacaaccctctcagccttcgctttgggaaacgctacaatggctacatttacagaagagctgttaaaagagccagaacaaaaaagttttcacccttctctctgttcttgcgagaactggaggtacccacctgaaacagaagactttctctggggaattatgttatttgtttggaaagtcaaactgtgacagcagtgttcttaaaactctttatttcagaaaaaaaggtttccctgattaaaacttttgcacctatctttaatgtaaaataattttcagatgctacaatggagagaactatgtaaataaaatacttttagagagctaaaaaaaaaaaaaaaaaaaaaaa

SEQ ID NO: 46 - protein, 169 aa, Oreochromis Niloticus
MRLLALAVVCALIAIQDGGSVGAALPGVDPAQRTHATGAVSSAFRRTAGDFLAEDPSLCFSLRENEDQRQLLCNDRRSNFNYNPLSLRFGKRYNGYIYRRAVKRARTKKFSPFSLFLRELEVPTNRRLSLGNYVICLESQTVTAVFLKLFISEKKVSLIKTFAPIFNVK

SEQ ID NO: 47 - protein, 390 aa, Oreochromis Niloticus
MYSSEELWNSTEQVWINGSGTNFSLGRHEDDEEEEGDKHPFFTDAWLBPLFFSLIMLVGLVGNSLVIYVISKHRQMRTATNFYIANLAATDIIFLVCCVPFTATLYPLPGWIFGNFMCKFVAFLQQVTVQATCITLTAMSGDRCYVTVYPLKSLRHRTPKVAMIVSICIWIGSFVLSTPILMYQRIEEGYWYGPRQYCMERFPSKTHERAFILYQFIAAYLLPVLTISFCYTLMVKRVGQPTVEPVDNNYQVNLLSERTISIRSKVSKMVVVIVLLFAICWGPIQIFVLFQSFYPNYQPNYATYKIKTWANCMSYANSSVNPIVYGFMGASFQKSFRKTFPFLFKHKVRDSSMASRTANAEIKFVAAEEGNNNNAVNSRSFNIRRIGTVF

SEQ ID NO: 48 - DNA, 1215 bp, Oreochromis Niloticus
aactcctgtcacgatgtactcctccgaggagctgtggaactccaccgagcaggtctggatcaacggatccggaacaaacttctctctaggaagacacgaggacgatgaggaggaggaaggagacaagcatcctttcttcacggatgcctggctggtccctctgttcttctctctcatcatgctggtcggactggtgggcaactctctggtcatttatgtcatttcaaaacacagacagatgaggacggcaaccaacttctacatagcaaacctggccgccactgacatcatcttcttggtgtgctgcgtccccttcaccgccaccctttaccccctccctggatggatctttggcaacttcatgtgcaaatttgtcgcctttctgcagcaggtgacggtgcaagccacctgcatcaccctgactgctatgagtggagaccgctgttacgtgacagrctaccccctgaaatctcttcggcaccgaacccccaaagtagccatgattgtcagcatttgcatttggatcggttctttcgtcctctctacaccaattttaatgtaccagcgtatagaggagggctactggtacggcccgaggcaatactgcatggagagatttccctctaagacgcatgagagggctttcatcctgtaccagttcattgctgcctacctgctgcctgtgctcactatctctttctgctacactctgatggttaagagggttggccagcccactgtagaacctgtagacaataattatcaggtgaatctcctgtctgagagaacaatcagcatccggagcaaagtttccaagatggtggtagtgattgtgcttctctttgccatctgctgggggcccatccagatctttgtcctcttccagtctttctatcctaactaccagcctaactacgccacatacaagatcaagacgtgggccaactgcatgtcctacgccaactcctctgtcaaccccatagtttatggcttcatgggagctagtttccaaaagtctttcaggaaaacttttcccttcctgttcaagcacaaggtcagagacagcagcatggcttcaaggactgccaatgctgagattaagtttgttgctgcggaggaaggcaacaataataacgcggtgaactgatcccgatcatttaacataagaaggatagggacagttttctaatgagaatcctgaaaaaaaaaaaaaaa

SEQ ID NO: 49 - DNA, 59 bp, Oreochromis Niloticus
tttcaactacaaccctctcagccttcgctttgggaaacgctacaatggctacatttaca

SEQ ID NO: 50 - DNA, 58 bp, Artificial Sequence
  Modified Allele
tttcaactacaaccctctcagccttcgctttgggannnnnnnnnaacgctacaatggc

SEQ ID NO: 51 - DNA, 59 bp, Artificial Sequence
  Modified Allele
tttcaactacaaccctctcagccttcgctttgggannnnaacgctacaatggctacatt

SEQ ID NO: 52 - DNA, 55 bp, Artificial Sequence
  Modified Allele
tttcaactacaaccctctcagccttcgctnnnacgctacaatggctacatttaca

SEQ ID NO: 53 - DNA, 48 bp, Artificial Sequence
  Modified Allele
tttcaactacaaccctctcagcctaacgctacaatggctacatttaca

SEQ ID NO: 54 - DNA, 47 bp, Artificial Sequence
  Modified Allele
tttcaactacaaccctctcagcctacgctacaatggctacatttaca

SEQ ID NO: 55 - DNA, 47 bp, Artificial Sequence
  Modified Allele
tttcaactacaaccctctcagccttcgctacaatggctacatttaca

SEQ ID NO: 56 - DNA, 47 bp, Artificial Sequence
  Modified Allele
tttcaactacgaccctctcagccttcgctacaatggctacatttaca

SEQ ID NO: 57 - DNA, 47 bp, Artificial Sequence
  Modified Allele
tttcaactacaaccctctcagccttcgctgcaatggctacatttaca

SEQ ID NO: 58 - DNA, 43 bp, Artificial Sequence
  Modified Allele
tttcaactacaaccctctcaacgctacaatggctacatttaca

SEQ ID NO: 59 - DNA, 64 bp, Oreochromis Niloticus
gacagtctaccccctgaaatctcttcggcaccgaacccccaaagtagccatgattgtcagcatt

SEQ ID NO: 60 - DNA, 61 bp, Artificial Sequence
  Modified Allele
gacagtctaccccctgaaatctcttcggcannnccccaaagtagccatgattgtcagcatt

SEQ ID NO: 61 - DNA, 59 bp, Artificial Sequence
  Modified Allele
gacagtctaccccctgaaatctcttcggcacccccaaagtagccatgattgtcagcatt

SEQ ID NO: 62 - DNA, 57 bp, Artificial Sequence
  Modified Allele
gacagtctaccccctgaaatctcttcggcccccaaagtagccatgattgtcagcatt

SEQ ID NO: 63 - DNA, 54 bp, Artificial Sequence
  Modified Allele
gacagtctaccccctgaaatctcttcccccaaagtagccatgattgtcagcatt

SEQ ID NO: 64 - DNA, 50 bp, Artificial Sequence
  Modified Allele
gacagtctaccccctgaaatnnnnccaaagtagccatgattgtcagcatt

SEQ ID NO: 65 - DNA, 38 bp, Artificial Sequence
  Modified Allele
gacagtctacccccaaagtacccatgattgtcagcatt

SEQ ID NO: 66 - DNA, 45 bp, Oreochromis Niloticus
ggcaccgaacccccaaagtagccatgattgtcagcatttgcattt

SEQ ID NO: 67 - DNA, 45 bp, Artificial Sequence
  Modified Allele
ggcacccaaagtagccatgattgtcagcatttgcatttggatcgg

SEQ ID NO: 68 - DNA, 51 bp, Oreochromis Niloticus
ggcaccgaacccccaaagtagccatgattgtcagcatttgcatttggatcg

SEQ ID NO: 69 - DNA, 48 bp, Artificial Sequence
  Modified Allele
ggcacccccaaagtagccatgattgtcagcatttgcatttggatcggt

SEQ ID NO: 70 - DNA, 51 bp, Artificial Sequence
  Modified Allele
ggcacccccncagtaaccatganttgattgatttcatttggattcggatcg

SEQ ID NO: 71 - DNA, 59 bp, Oreochromis Niloticus; identical to SEQ ID NO: 49

SEQ ID NO: 72 - DNA, 47 bp, Artificial Sequence; identical to SEQ ID NO: 55
  Modified Allele

SEQ ID NO: 73 - DNA, 41 bp, Artificial Sequence
  Modified Allele
tttcaactacaaccctctcagctacaatggctacatttaca

SEQ ID NO: 74 - DNA, 64 bp, Oreochromis Niloticus; identical to SEQ ID NO: 59

SEQ ID NO: 75 - DNA, 61 bp, Artificial Sequence
  Modified Allele
gacagtctaccccctgaaatctcttcggcaaacccccaaagtagccatgattgtcagcatt

SEQ ID NO: 76 - DNA, 59 bp, Artificial Sequence; identical to SEQ ID NO: 61
  Modified Allele

SEQ ID NO: 77 - DNA, 57 bp, Artificial Sequence
  Modified Allele
gacagtctaccccctgaaatctcttcggcacccaaagtagccatgattgtcagcatt

SEQ ID NO: 78 - DNA, 56 bp, Artificial Sequence
  Modified Allele
gacagtctaccccctgaaatctcttcggcaccaaagtagccatgattgtcagcatt

SEQ ID NO: 79 - DNA, 55 bp, Artificial Sequence
  Modified Allele
gacagtctaccccctgaaatctcttcggcacaaagtagccatgattgtcagcatt

SEQ ID NO: 80 - DNA, 53 bp, Artificial Sequence
  Modified Allele
gacagtctaccccctgaaatctcttcggcaaagtacccatgattgtcagcatt

SEQ ID NO: 81 - DNA, 74 bp, Oreochromis Niloticus
agaagtaatttcaactacaaccctctcagccttcgctttgggaaacgctacaatggctacatttacagaagagc

SEQ ID NO: 82 - protein, 25 aa, Artificial Sequence
  Modified Protein
RSNFNYNPLSLRFGKRYNGYIYRRA

SEQ ID NO: 83 - protein, 19 aa, Artificial Sequence
  Modified Protein
RSNFNYNPLSYNGYIYRRA

SEQ ID NO: 84 - DNA, 63 bp, Oreochromis Niloticus
acagtctaccccctgaaatctcttcggcaccgaacccccaaagtagccatgattgtcagcatt

SEQ ID NO: 85 - protein, 25 aa, Artificial Sequence
  Modified Protein
RSNFNYNPLSLRFGKRYNGYIYRRA

SEQ ID NO: 86 - protein, 12 aa, Artificial Sequence
  Modified Protein
TVYPLKSLRPPK